(19) United States
(12) Patent Application Publication
(10) Pub. No.: US 2025/0229062 A1
(43) Pub. Date: Jul. 17, 2025

(54) CATHETER CLAMPING DEVICE

(71) Applicant: EDWARDS LIFESCIENCES CORPORATION, Irvine, CA (US)

(72) Inventors: Mark Andrew Williams, Newport Beach, CA (US); James Richard Raven, JR., Murfreesboro, TN (US); John X. Wang, Orange, CA (US); Bryan A. Janish, Huntington Beach, CA (US)

(21) Appl. No.: 19/060,666

(22) Filed: Feb. 22, 2025

Related U.S. Application Data
(63) Continuation of application No. PCT/US2023/030052, filed on Aug. 11, 2023.
(60) Provisional application No. 63/373,389, filed on Aug. 24, 2022.

(51) Int. Cl. A61M 25/10

(52) U.S. Cl. CPC A61M 25/10; A61M 2205/0216; A61M 2205/58

(57) ABSTRACT

A clamping device for a catheter shaft can include a first member having a first indicator surface and defining a first inner bore having a non-circular cross-sectional profile, and a second member having a second indicator surface, and an extending annular element defining a second inner bore, the annular element received within the first inner bore and comprising an outer surface having a non-circular cross-sectional profile. The clamping device can be movable between a release state in which the second inner bore has a first diameter, and a clamped state in which the second inner bore has a second diameter less than the first diameter.

### CROSS-REFERENCED TO RELATED APPLICATIONS

This application is a continuation of International Application No. PCT/US2023/030052, filed Aug. 11, 2023, which claims the benefit of U.S. Patent Application No. 63/373,389, filed Aug. 24, 2022, the entire disclosures all of which are incorporated by reference for all purposes.

### FIELD

This disclosure relates generally to clamping and/or locking devices and methods for clamping and/or locking catheter assemblies.

### BACKGROUND

Catheter assemblies can be used for a variety of interventional procedures including aortic occlusion, angioplasty, urinary catheterization, nephrostomy, hemodialysis, medical device implantation, etc. In many procedures a distal end portion of the catheter assembly is positioned at a selected site within the body and maintained at that site for a selected time or throughout the duration of the procedure.

Methods and devices have been developed for mitigating or preventing movement of the distal end portion of the catheter assembly away from the selected site. However, such devices can be unwieldly or difficult for a physician to actuate during a catheterization procedure. Accordingly, a need exists for improved devices and methods for mitigating or preventing movement of a catheter assembly.

### SUMMARY

Described herein are examples of a clamping and/or locking device for use with a catheter assembly, as well as methods for using the same. The clamping device can be used to secure a catheter against movement relative to one or more other objects, including introducer assemblies, or other catheters.

In a representative example, a clamping device for a shaft of a catheter can comprise a first member comprising a first main body having a first indicator surface, the first main body defining a first inner bore having a non-circular cross-sectional profile, and a second member comprising a second main body having a second indicator surface and an annular element extending from a first end portion of the main body and defining a second inner bore, the annular element received within the first inner bore and comprising an outer surface having a non-circular cross-sectional profile. The clamping device can be movable between a first, release state in which the second member is in a first rotational position relative to the first member and the second inner bore has a first diameter, and a second, clamped state in which the second member is in a second rotational position relative to the first member and the second inner bore has a second diameter less than the first diameter. When the clamping device is in the clamped state the first and second indicator surfaces are rotationally aligned with one another, and when the clamping device is in the release state the first and second indicator surfaces are rotationally offset from one another.

In a representative example, a catheter assembly can comprise a shaft and a clamping device disposed on the shaft. The clamping device can comprise a first member comprising a first main body having a first indicator surface, the first main body defining a first inner bore having a non-circular cross-sectional profile, and a second member comprising a second main body having a second indicator surface and an annular element extending from a first end portion of the main body, the annular element having a non-circular cross-sectional outer profile corresponding to the non-circular cross-sectional profile of the first inner bore, the second member defining a second inner bore extending through the annular element, the shaft extending through the second inner bore. The annular element can be received within the first inner bore. the clamping device can be movable between a first, release state in which the second member is in a first rotational position relative to the first member and the second inner bore has a first diameter, and a second, clamped state in which the second member is in a second rotational position relative to the first member and the second inner bore has a second diameter less than the first diameter. When the clamping device is in the clamped state the first and second indicator surfaces are rotationally aligned with one another, and wherein when the clamping device is in the release state the first and second indicator surfaces are rotationally offset from one another.

In another representative example, a clamping device can include a first member comprising a first main body having a first indicator surface and an axially-extending protrusion, the main body defining a first inner bore extending therethrough, and a second member comprising a second main body having a second indicator surface, a recess within which the axially-extending protrusion of the first member is disposed, and an annular element extending from a first end portion of the main body axially away from a second end portion of the main body and into the first inner bore, the annular element defining a second inner bore extending therethrough. The clamping device can be movable between a first, release state in which the second inner bore has a first diameter, and a second, clamped state wherein the second inner bore has a second diameter smaller than the first diameter. The recess can comprise a first stopping surface and a second stopping surface and wherein when the first and second members are in the release state the axially-extending protrusion abuts the first stopping surface and when the first and second members are in the clamped state the axially-extending protrusion abuts the second stopping surface.

In another representative example, a catheter assembly can comprise a shaft and a clamping device disposed on the shaft. The clamping device can comprise a first member comprising a first main body having a first indicator surface an axially-extending protrusion, the main body defining a first inner bore extending therethrough, and a second member fixed axially relative to the first member and comprising a second main body having a second indicator surface, a recess within which the axially-extending protrusion of the first member is disposed, and an annular element extending from a first end portion of the main body axially away from a second end portion of the main body and into the first inner bore, the annular element defining a second inner bore extending therethrough, the shaft extending through the second inner bore. The clamping device can be movable between a first, release state in which the second inner bore has a first diameter, and a second, clamped state wherein the second inner bore has a second diameter smaller than the first diameter. The recess can comprise a first stopping surface and a second stopping surface and wherein when the first and second members are in the release state the axially-extending protrusion abuts the first stopping surface and when the first and second members are in the clamped state the axially-extending protrusion abuts the second stopping surface.

In still another representative example, a clamping device can comprise a first member comprising a first main body having a first indicator surface and defining a first inner bore extending through the main body, and a second member fixed axially relative to the first member and comprising a second main body having a second indicator surface and an annular element extending from a first end portion of the main body axially away from a second end portion of the main body and into the first inner bore, the annular element defining a second inner bore extending therethrough. The clamping device can be movable between a first, release state in which the second inner bore has a first diameter, and a second, clamped state wherein the second inner bore has a second diameter smaller than the first diameter. When the clamping device is in the clamped state the first and second indicator surfaces are rotationally aligned with one another, and wherein when the clamping device is in the release state the first and second indicator surfaces are rotationally offset from one another.

In a representative example, a method can comprise disposing a clamping device in a release state on a shaft such that the shaft extends through the clamping device, wherein when in the release state a first indicator surface and a second indicator surface of the clamping device are rotationally offset from one another, and moving the clamping device along the shaft to a selected position. The method can further comprise rotating a first member of the clamping device relative to a second member of the clamping device to move the clamping device from the release state to a clamped state such that the clamping device is restrained from movement relative to the shaft and such that the first and second indicator surfaces are rotationally aligned with one another, the first member comprising a first main body including the first indicator surface and defining a first inner bore having a non-circular cross-sectional profile, and the second member comprising a second main body including the second indicator surface and an annular element extending from a first end portion of the main body and defining a second inner bore, the annular element received within the first inner bore and comprising an outer surface having a non-circular cross-sectional profile.

In another representative example, a method can comprise sterilizing any of the devices or assemblies described herein.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of an exemplary catheter assembly configured as an antegrade cardioplegia delivery catheter.

FIG. 2 is a side view of the distal end of the delivery catheter of FIG. 1.

FIG. 3 is a cross-sectional view of a shaft of the delivery catheter of FIG. 1.

FIG. 4 is a perspective cross-sectional view of an atraumatic tip of the delivery catheter of FIG. 1.

FIGS. 5-7 are partial cross-sectional views of the distal end of a catheter assembly configured as an antegrade cardioplegia delivery catheter used to occlude a portion of the ascending aorta.

FIG. 8 is a perspective view of an exemplary catheter assembly configured as an antegrade cardioplegia delivery catheter including an exemplary clamping device.

FIG. 9 is a perspective view of the clamping device of FIG. 8.

FIG. 10 is a side elevation view of an exemplary catheter assembly including an exemplary clamping device and an introducer.

FIGS. 11-12 are perspective views of an exemplary clamping device, shown in the release state.

FIG. 13 is an exploded perspective view of the clamping device of FIG. 11.

FIG. 14 is a perspective view of a first member of the clamping device of FIG. 11.

FIG. 15 is an end view of the first member of the clamping device of FIG. 14.

FIG. 16 is a perspective view of a first member of the clamping device of FIG. 11.

FIG. 17 is a perspective view of a cap member of the clamping device of FIG. 11.

FIG. 18 is a side elevation view of the cap member of FIG. 17.

FIG. 19 is a perspective view of the cap member of FIG. 17.

FIG. 20 is a perspective view of a second member of the clamping device of FIG. 11.

FIGS. 21-22 are end views of the second member of the clamping device of FIG. 20.

FIG. 23 is a perspective view of a portion of the second member of the clamping device of FIG. 11.

FIG. 24 is a perspective view of an elastomeric member of the clamping device of FIG. 11.

FIG. 25 is a perspective view of the second member of the clamping device of FIG. 20 including the elastomeric member of FIG. 24.

FIG. 26 is a transverse cross-sectional view of the clamping device of FIG. 11 along line A-A where the clamping device is in the release state.

FIG. 27 is a transverse cross-sectional view of the clamping device of FIG. 11 where the clamping device is in the clamped state.

FIG. 28 is a top down view of the clamping device of FIG. 11 shown in the clamped state.

FIG. 29 is a longitudinal cross-sectional view of the clamping device of FIG. 11.

FIGS. 30-31 are perspective views of the clamping device of FIG. 11 including another example of a cap member.

### DETAILED DESCRIPTION

### General Considerations

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

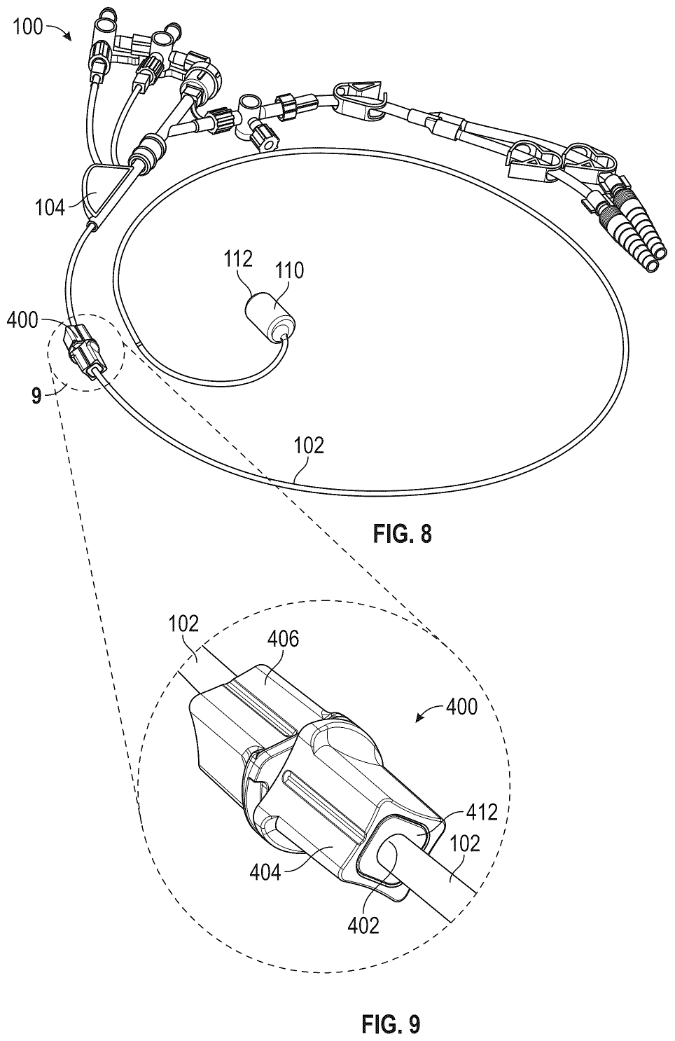

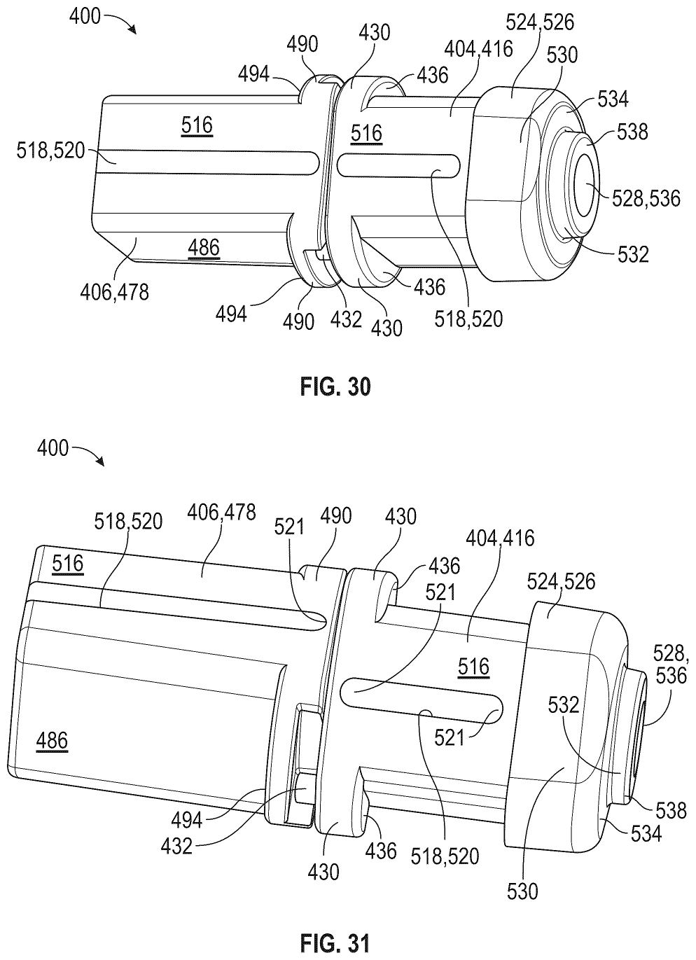

All features described herein are independent of one another and, except where structurally impossible, can be used in combination with any other feature described herein. For example, a delivery apparatus 100 as shown in FIG. 8 can be used in combination with the clamping device 400 shown in FIGS. 30-31 and described herein. In other examples, the clamping devices described herein can be used with any catheter assembly.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

In some examples, values, procedures, or apparatus may be referred to as “lowest,” “best,” “minimum,” or the like. It will be appreciated that such descriptions are intended to indicate that a selection among many alternatives can be made, and such selections need not be better, smaller, or otherwise preferable to other selections.

In the description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

### Examples of the Disclosed Technology

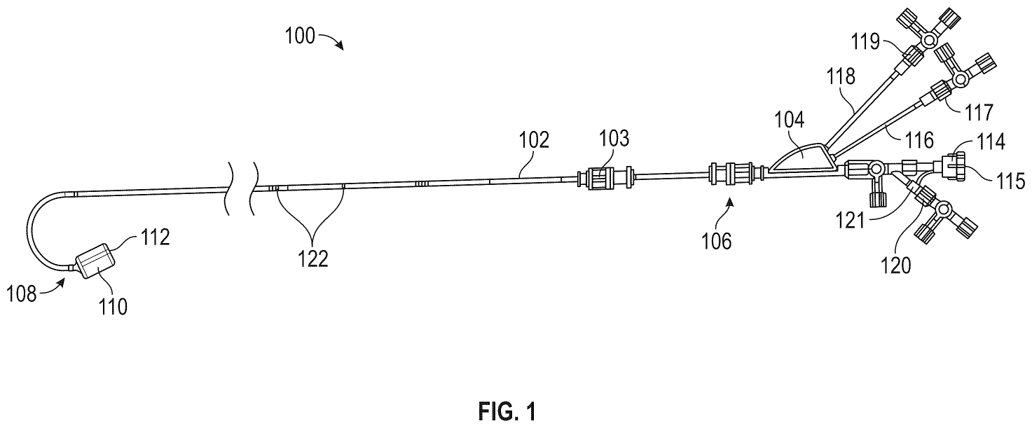

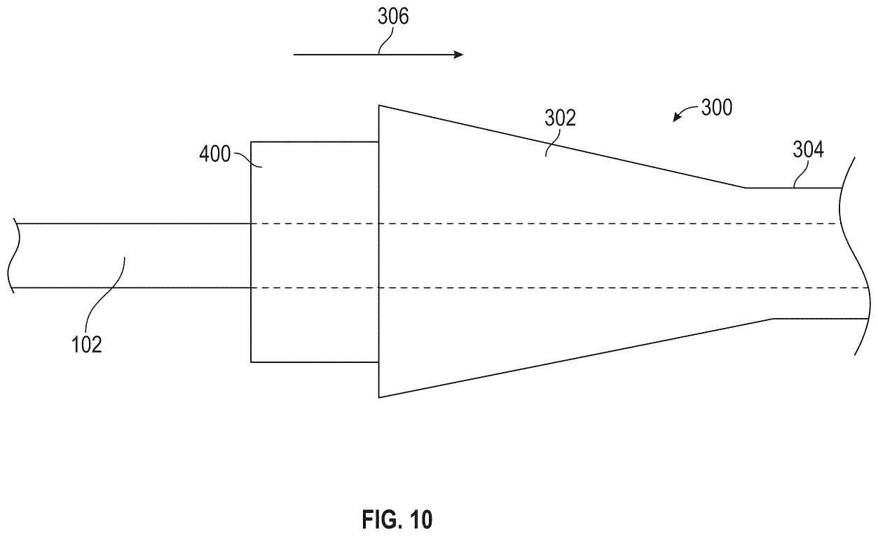

An exemplary example of a catheter assembly configured as an antegrade cardioplegia delivery catheter 100 is illustrated in FIG. 1. A distal end of the delivery catheter 100 can be inserted into a patient using, for example, an introducer assembly (see e.g., introducer assembly 304 in FIG. 10) comprising an introducer cannula and a hemostasis valve. The hemostasis valve can be configured to allow passage of the delivery catheter 100 therethrough.

As shown in FIG. 1, in the illustrated example, the delivery catheter 100 includes a catheter shaft 102 that can be inserted into a patient and located at a desired location, such as within the ascending aorta of the patient. Accordingly, the shaft 102 may have a length such that when a distal end 108 of the shaft 102, including an expandable member 110, is positioned at a desired location within the patient, a proximal end 106 of the shaft 102 may remain exterior to the patient. The proximal end 106 of the shaft 102 may be positioned, for instance, adjacent a peripheral access site, such as in the femoral artery to facilitate a minimally invasive procedure. A hub 104 may also be attached to the shaft 102. The hub 104 may serve any number of purposes. For instance, in the example shown in FIG. 1, the hub 104 may have various extension arms 114, 116, 118, 120 that serve various purposes. Such extension arms may, for instance, facilitate expansion of the expandable member 110, delivery of cardioplegic fluid, monitoring of pressure or characteristics within the vasculature at the distal tip 112, insertion of guidewires, stents, replacement valves, other devices or components, or any combination of the foregoing. Extension arm 114 can comprise a rotating hemostasis valve, which can be connected to the central lumen of catheter shaft 102 to allow for guidewire insertion, fluid injection (e.g., cardioplegia delivery), and aortic root venting.

The delivery catheter 100 can also include a clamping device, for example, clamping device 103 disposed on the shaft 102 that can be used to retain the distal end 108 of the shaft 102 including the expandable member 110 at a selected position within the body of a patient, as described in more detail below.

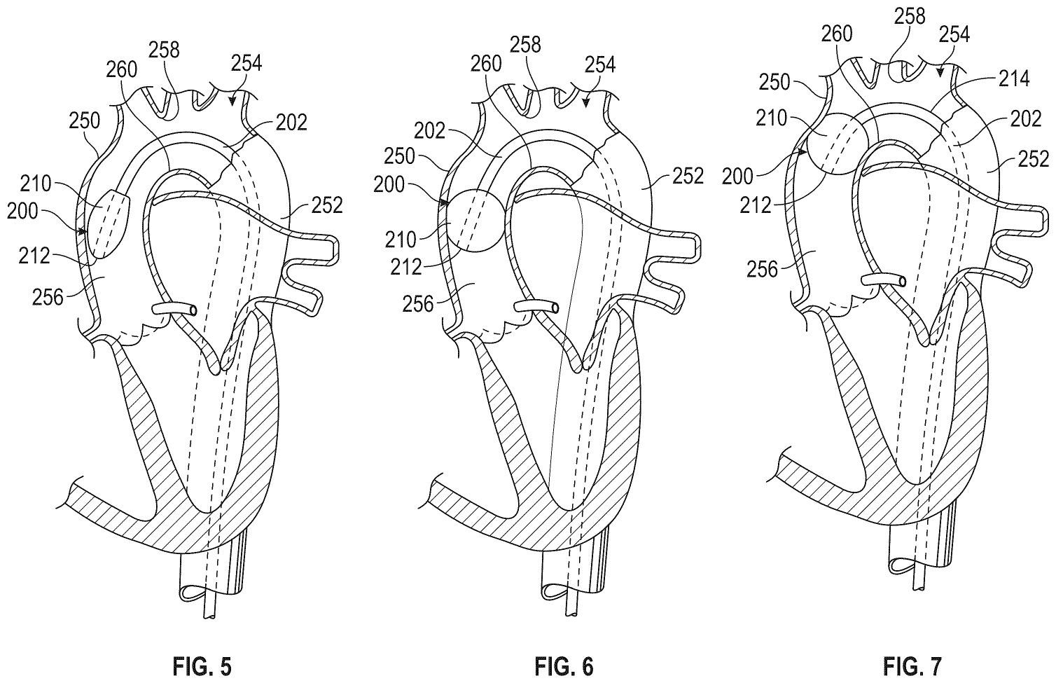

The delivery catheter 100 may be used to occlude a portion of a patient's vasculature at or near the heart, while also supplying cardioplegic fluid to the heart. An exemplary manner in which the delivery catheter 100 can be used to occlude vasculature may be understood particularly with reference to FIGS. 5-7.

The delivery catheter 100 may include an expandable member 110, which can be, for example, an expandable balloon. The expandable member 110 may be generally positioned at the distal end 108 of the shaft 102, and may be proximate or adjacent a distal tip 112 of the shaft 102. The expandable member 110 may be configured to vary its size, diameter, or other dimension in any suitable manner. The expandable member 110 may be formed of a flexible material, for instance, be polyurethane, PTFE, or other material that is blow-molded, dip-molded, or otherwise formed. The expandable member 110 may also be formed of other materials, formed in other manners, or take other forms. For instance, the expandable member 110 need not be a balloon, and could be any other suitable type of selectively expandable element.

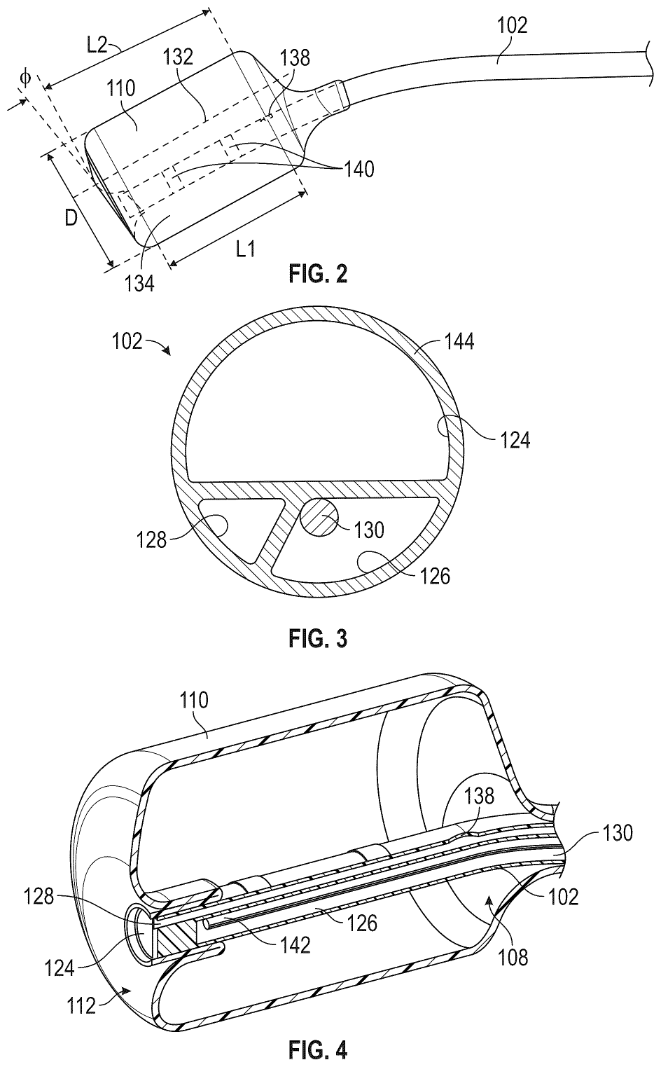

The expandable member 110 of FIGS. 2 and 4 is illustrated in an expanded state. It should be appreciated, however, that the expandable member 110 may be inserted into a patient while in a collapsed, partially collapsed, or other state that may allow the expandable member 110 to pass more easily through the patient's vasculature. In some examples, as the expandable member 110 moves through the vasculature, the expandable member 110 does not substantially occlude the vasculature, at least not until the distal tip 112 of the shaft 102 is at or near an intended location. Once at the intended location, the expandable member 110 may be expanded.

Expansion of the expandable member 110 may be performed in any suitable manner. For instance, where the expandable member 110 is an expandable balloon, a fluid may be selectively passed through the shaft 102 and into the expandable member 110. In FIG. 1, for instance, the shaft 102 may connect to the hub 104, and may be in fluid communication with one or more of the various extension arms 114, 116, 118, 120. The shaft 102 may have one or more lumens therein to receive fluid, instruments, or other items. For instance, as shown in FIGS. 3 and 4, the shaft 102 may have a multi-lumen design. Each of the multiple lumens 124, 126, 128 may be in communication with the one or more extension arms 114, 116, 118, 120 (FIG. 1) with access ports 115, 117, 119, 121, which may act as access ports to the respective lumens 124, 126, 128.

In the illustrated example, the shaft 102 may include a primary lumen 124 and multiple secondary lumens 126, 128. The secondary lumen 126 may, for instance, extend along a length of the shaft 102 and terminate at a location within the expandable member 110. As shown in FIG. 4, for instance, the secondary lumen 126 may terminate near the distal tip 112 of the shaft 102. The secondary lumen 126 may be in fluid communication with an inflation port 138 that extends through a sidewall of the shaft 102. The inflation port 138 can be within the expandable member 110, such that as fluid is inserted through the lumen 126 and exits the shaft 102 through the inflation port 138, the expandable member 110 may inflate or otherwise expand. Conversely, fluid dispelled from the expandable member 110 may pass through the inflation port 138 and into the shaft 102 as the expandable member 110 contracts.

The expandable member 110 may have any number of suitable constructions or configurations. For instance, in FIG. 4, the expandable member 110 is illustrated as an inflated balloon having a generally elongated, hexagonal side profile, and with the shaft 102 being eccentric relative to the central axis 132 of the expandable member 110. The particular dimensions and configuration of the expandable member 110 can vary as desired to, for example, occlude an ascending aorta of a patient. The eccentric profile of the shaft 102 may provide differing sizes of portions 134, 136; however, the general shape of the expandable member 110 may additionally or alternatively be varied. Further details of the expandable member 110 can be found, at least, in U.S. patent application Ser. No. 17/650,239, filed Feb. 7, 2022.

The expandable member 110 is but one example of a suitable expandable member, and other expandable members may be used. For instance, in other examples, the expandable member 110 may be spherical, trapezoidal, cylindrical, barrel-shaped, or otherwise configured.

To facilitate cardioplegic functions of the delivery catheter 100, the delivery catheter 100 may allow cardioplegic fluid to be passed from a fluid source or reservoir and into the ascending aorta or other location within a patient. FIGS. 3 and 4 illustrate a particular manner in which such features can be provided. For instance, as noted previously, the shaft 102 optionally includes multiple fluid conduits, channels, lumens, or other features. In particular, in the illustrated example, the shaft 102 may include a primary lumen 124 that is optionally in fluid communication with an extension arm 114 that acts as a port to allow the introduction of cardioplegic fluid, guidewires, surgical instruments, or other elements. Cardioplegic fluid may be pressurized and passed through the lumen 124 towards the distal tip 112 of the shaft 102. As best shown in FIG. 4, the distal end of the shaft 102 may include an opening generally corresponding to the lumen 124. For instance, the lumen 124 may be open at the distal tip 112 such that the pressurized cardioplegic fluid exits the shaft 102 distal to the expandable member 110.

The delivery catheter 100 may provide still other features and uses. For instance, cardiac and/or vascular characteristics can be monitored using the delivery catheter 100. Such characteristics may include, for instance, flow rates, beat rates (if any), pressure, or dimensions, or other characteristics. In one example, such as where the delivery catheter 100 is configured to occlude the ascending aorta, the delivery catheter 100 may be adapted to measure a pressure within the aorta, such as the aortic root pressure. As shown in FIGS. 3 and 4, for instance, a secondary lumen 128 may extend to a vent at or near the distal tip 112 of the shaft 102. The secondary lumen 128 may be in fluid communication with a pressure monitoring device (e.g., through a connection at extension arm 118 of FIG. 1), thereby allowing root aortic pressure to be monitored throughout a surgical or other procedure.

The delivery catheter 100 may be configured to provide any number of features. In accordance with some examples, the shaft 102 may be adapted to provide still other features and aspects. For instance, as shown in FIG. 1, the shaft 102 may include one or more markings 122 thereon. Such markings may be bands, ink, radiopaque markers, or otherwise structured to facilitate visualization inside or outside the patient. For instance, in one example, the markings 122 are radiopaque markings that are visible under transesophageal echocardiography visualization or other visualization techniques, so as to facilitate positioning of the shaft within a patient. Where an expandable member 110 is to be placed at a particular location, the expandable member 110 may optionally include additional markings (e.g., platinum-iridium and/or tungsten markers) to facilitate visualization. For instance, as best shown in FIG. 2, one or more markings 140 may be placed on, within, or proximate the expandable member 110 to thereby allow identification of a position of the expandable member 110 when a particular visualization technique is used.

The shaft 102 may be otherwise structured to facilitate insertion, removal, and/or placement of the delivery catheter 100 during a surgical procedure. For instance, as shown in FIGS. 3 and 4, the shaft 102 may include two components. Such components include, in this example, a body element 144 and a core element 130. The body element 144 may, for instance, generally define the shape of the shaft 102 and the lumens 124, 126, 128 within the shaft. In one example, the body element 144 may be formed of any suitable material and using any number of different manufacturing processes. For instance, the body element 144 may be formed from a flexible material that can bend as the shaft 102 translates through a patient's vasculature, to thereby match contours within the patient's body. Suitable materials may include, for instance, ethylene tetrafluoroethylene (ETFE) or polytetrafluoroethylene (PTFE). In another example, the body element 144 is formed from another suitable biocompatible material or biocompatible polymer, such as polyether block amide (for example, PEBAX® polyether block amide (Arkema, Colombes, France). In at least one example, such as that shown in FIGS. 3 and 4, the shaft 102 may include a core 130 within the secondary lumen 126. The secondary lumen 126 may, as discussed previously, be used for facilitating expansion of the expandable member 110, or for any other desired feature. The core 130 may be a wire extending along all or a portion of the length of the shaft 102. The core 130 may have a stiffness and strength that provides additional column stiffness to facilitate placement of the shaft 102. The core 130 may additionally, or alternatively, provide kink resistance or define a desired shape of the shaft 102.

For instance, as reflected in FIG. 1, the distal end 108 of the shaft 102 may have a bend, curve, or other shape. In some examples, the shaft 102 may be configured to pass through the descending aorta and into the ascending aorta. To do so, the curved distal end 108 may pass around a relatively tight curve radius, namely the curve radius defined by the aortic arch.

While curvature of the distal end 108 may be produced by allowing the body clement 144 and/or core 130 to be made of a flexible material, in other examples the core is pre-designed and manufactured to maintain a specific curved profile. In still other examples, such curved profile may be selectively activated in the shaft 102. To obtain these and other characteristics, in one example, the core 130 can be comprised of biocompatible materials that are at least temporarily deformable. Suitable biocompatible materials can include, for example, superelastic and/or shape memory materials (e.g., copper-zinc-aluminum; copper-aluminum-nickel; nickel-titanium alloys known as nitinol; cobalt-chromium-nickel alloys, cobalt-chromium-nickel-molybdenum alloys, nickel-titanium-chromium alloys, and the like). In addition, other suitable materials may include stainless steel, silver, platinum, tantalum, palladium, cobalt-chromium alloys, niobium, iridium, any equivalents thereof, alloys thereof or combinations thereof. Further details of the shaft curvature and examples of the core can be found, for example, in U.S. Pat. No. 10,130,371, which is incorporated herein by reference in its entirety for all purposes.

In some examples, the core 130 may be a wire, although the core 130 may take other forms. As illustrated in FIG. 4, the core 130 may be a wire having a variable cross-sectional shape. In particular, in at least one example, the core 130 may have a distal end 142 at least proximate the distal end 108 of the shaft 102. As the core 130 approaches the distal tip 112 of the shaft 102, the size of the core 130 may, in some examples, decrease, such as by having a tapered, stepped, or other configuration. In such a manner, the strength of the core 130 at the distal tip 112 may be decreased, thereby also reducing the force that the core 130 can exert at the distal tip 112. With reduced force at the distal tip 112, trauma to a patient's vasculature may be decreased.

The shaft 102 and the hub 104 may be formed in any number of manners, or have any other number of features or configurations. For instance, the size of the shaft 102 may be varied as desired. In accordance with one example, the shaft 102 may have an outer diameter of between about eight and ten French, so as to be passable from a peripheral artery through the descending aorta, and into the ascending aorta as described herein. Depending on other uses of the delivery catheter 100, the patient with whom the catheter 100 is used, or other factors, the size of the shaft 102 may be larger than ten French, or smaller than eight French. Further details of the shaft and hub can be found, at least, in U.S. patent application Ser. No. 17/650,239, filed Feb. 7, 2022, the entire disclosure which is incorporated by reference for all purposes.

Referring now to FIGS. 5-7, an exemplary catheter assembly configured as an aortic occlusion assembly 200 can be used to occlude a selected occlusion site (e.g., a patient's aorta 250) in the following exemplary manner. FIGS. 5-7 generally illustrate a process of inserting a shaft 202 and expandable member 210 of a delivery catheter into a patient's aorta 250, expanding the expandable member 210, and retracting the expandable member 210 to secure the expandable member 210 in an occluding position. FIGS. 5-7 represent one method of using the delivery catheter 100. Thus, in one example, the shaft 202 corresponds to the shaft 102 and the expandable member 210 corresponds to the expandable member 110.

More particularly, in FIG. 5, a shaft 202 and expandable member 210 may be passed through the descending aorta 252, around the aortic arch 254, and into the ascending aorta 256. During such movement, the expandable member 210 may be in a deflated or otherwise contracted state. In order to facilitate placement of the expandable member 210 and a distal tip 212 within the ascending aorta 256, the shaft 202 may be flexible. In particular, the shaft 202 may bend to generally correspond to a curve of the aortic arch 254. For instance, the aortic arch 254 may have an upper profile 258 and a lower profile 260. The shaft 202 may bend so as to generally have a curve that extends partially between the upper and lower profiles 258, 260 of the aortic arch 254.

The expandable member 210 and distal tip 212 may be located using any suitable visualization technique. Once positioned in the desired location, the expandable member 210 may be expanded using any suitable manner, including those described herein. For instance, the expandable member 210 may be a balloon that is inflated to substantially occlude the ascending aorta 256. In FIG. 6, for instance, the expandable member 210 has a generally spherical shape and the shaft 202 is generally concentric within the expandable member 210.

Inflation of the expandable member 210 on the distal end of the shaft 202 can fix the distal tip 212 of the shaft 202 within the ascending aorta 256 and isolate the left ventricle of the heart and the upstream portion of the ascending aorta 256 from the rest of the arterial system downstream from the expandable member 210. The passage of any debris or emboli, solid or gaseous, generated during a cardiovascular procedure to regions downstream from the site can be substantially prevented by the expanded expandable member 210. Fluid containing debris or emboli can be removed from the region between the aortic valve and the occluding expandable member 210 through an interior lumen of the shaft 202. A clear, compatible fluid (e.g., an aqueous based fluid such as saline) delivered through an interior lumen or the cardioplegic fluid may be maintained in the region wherein the cardiovascular procedure is to be performed to facilitate use of an angioscope or other imaging means that allows for direct observation. Such use of a delivery catheter may be particularly useful in the removal of an aortic heart valve and replacement thereof with a prosthetic heart valve which procedure is described in U.S. Pat. No. 5,738,652, which is incorporated herein by reference in its entirety for all purposes.

The expandable member 210 may have forces applied thereto that cause the expandable member 210 to shift position. For instance, as cardioplegic fluid is expelled from the distal tip 212 the fluid flow may generally cause the expandable member 210 to move upward through the ascending aorta 256 and towards the aortic arch 254. Other forces may also be applied, for instance, a decrease in perfusion pressure may also cause the expandable member 210 to move towards the aortic arch 254. In contrast, the systemic blood pressure, increases in root vent suction, or increases in perfusion pressure may tend to cause the expandable member 210 to move further into the ascending aorta 256 and away from the aortic arch 254.

Migration of the expandable member 210 may be particularly likely where slack is present in the shaft 202. Accordingly, to minimize migration of the expandable member 210, a surgeon may pull on the delivery catheter so as to at least partially retract the shaft 202. For instance, a surgeon may pull two to three inches of slack out of the shaft 202. As a result, the expandable member 210 may move towards the aortic arch 254. In retracting the expandable member 210, external surfaces of the expandable member 210 may also more fully engage the upper and lower portions of the ascending aorta 256, thereby more securely positioning the expandable member 210 as it occludes the aorta.

As shown in FIG. 7, the shaft 202 may have a curved profile 214 that generally corresponds to a portion of the aortic arch 254. In this example, for instance, the curved profile 214 allows the shaft 202 to curve around the aortic arch 254 generally between the upper profile 258 and lower profile 260 of the aortic arch 254. The shaft 202 may be generally mid-way between the upper and lower profiles 258, 260, although such is not necessary. For instance, the shaft 202 may be generally flexible such that the profile 214 adapts to a suitable geometry that allows the expandable member 210 to remain at the illustrated occluding position.

When the slack is pulled from the shaft 202, such that the expandable member 210 is secured within the ascending aorta 256, the distal tip 212 of the shaft 202 may migrate and change orientation within the ascending aorta 256. More particularly, in the illustrated example, the distal tip 212 may be positioned at an angle relative to the ascending aorta 256. As noted herein, cardioplegic fluid may, in some instances, be perfused to the ascending aorta 256 through the distal tip 212. Generally speaking, the shape of the expandable member 210, curvature of the shaft 202, and location of the shaft 202 within the expandable member 210 may each contribute to the orientation of the distal tip 212.

Once the expandable member (e.g., expandable member 110 or 210) is positioned at a selected position within the body (e.g., within the ascending aorta 256), the physician can engage a clamping device, such as clamping device 103 of delivery catheter 100, to prevent or minimize movement of the expandable member relative to the selected position and movement of the shaft relative to the aorta.

As shown in FIG. 8, an exemplary clamping device 100 can be used with a delivery catheter, such as delivery catheter 100 described above. A clamping device can be used to prevent movement of an expandable member (e.g., expandable member 110) relative to a patient's aorta. For instance, as shown in FIG. 10, a clamping device 400 can be disposed on the shaft 102 of the delivery catheter 100 such that, when in the clamped or locked state, it abuts a proximal end of a hub 302 of an introducer assembly 300, thereby preventing axial movement of the shaft 102 relative to the hub 302. Though some of the below examples are described with respect to an antegrade cardioplegia delivery catheter, it should be understood that the clamping devices described herein can be used with any catheter assembly.

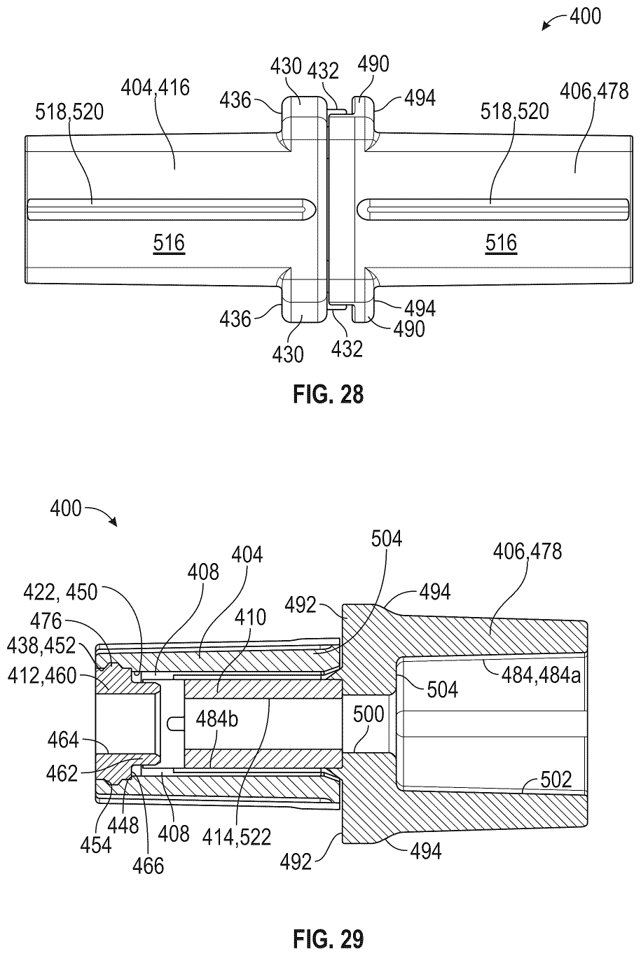

Referring now to FIG. 9, the shaft 102 of the delivery catheter 100 can extend through an inner bore 402 of the clamping device 400. The clamping device 400 can be movable between an unclamped or release state (FIG. 9) wherein the clamping device can be moved axially along the length of the shaft 102 and/or rotated relative to the shaft 102, and a clamped state (also referred to as a locked state; FIG. 28) wherein the clamping device 400 engages an outer surface of the shaft, thereby preventing movement of the clamping device 400 and the shaft 102 relative to one another.

Referring again to FIG. 10, in use, an elongated sheath 304 of the introducer assembly 300 is inserted into a blood vessel of the patent (e.g., a femoral artery) and the delivery catheter 100 is inserted through the introducer assembly 300 into the blood vessel. The diameter of the clamping device 400 is greater than the diameter of a proximal opening of the hub 302, preventing the portion of the shaft 102 on which the clamping device 400 is mounted from passing through the introducer 300 and into the patient's body when the clamping device is in the clamped state. The clamping device 400 (shown in a simplified version in FIG. 10) can bear against the introducer assembly and thereby mitigate movement of the shaft 102 relative to the introducer assembly 300 in the distal direction 306. Mitigating movement of the shaft 102 relative to the introducer assembly 300 thereby mitigates movement of the distal end portion of the shaft 102 within the patient's body.

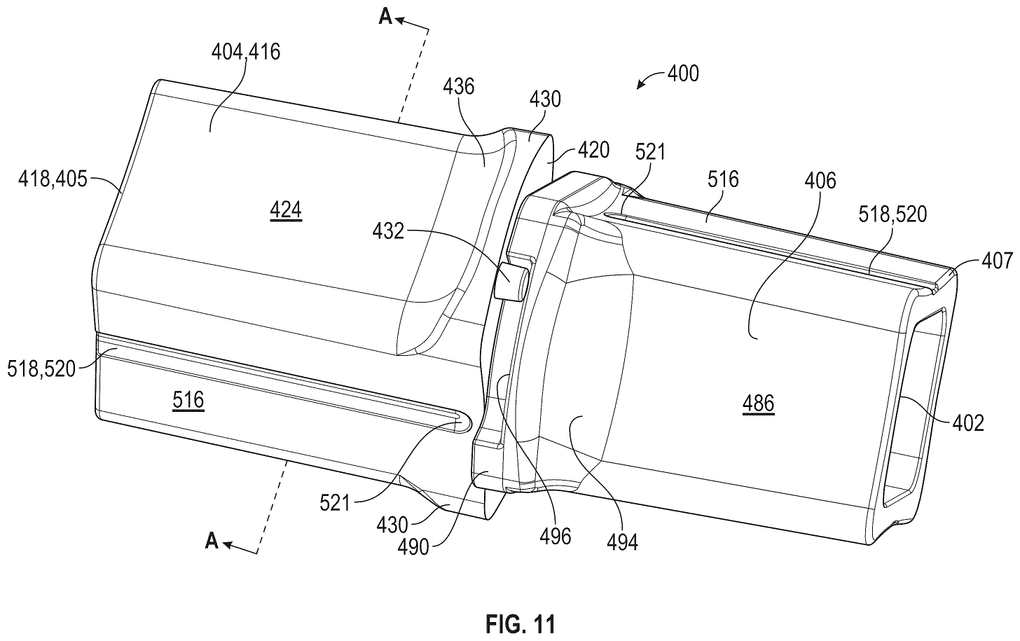

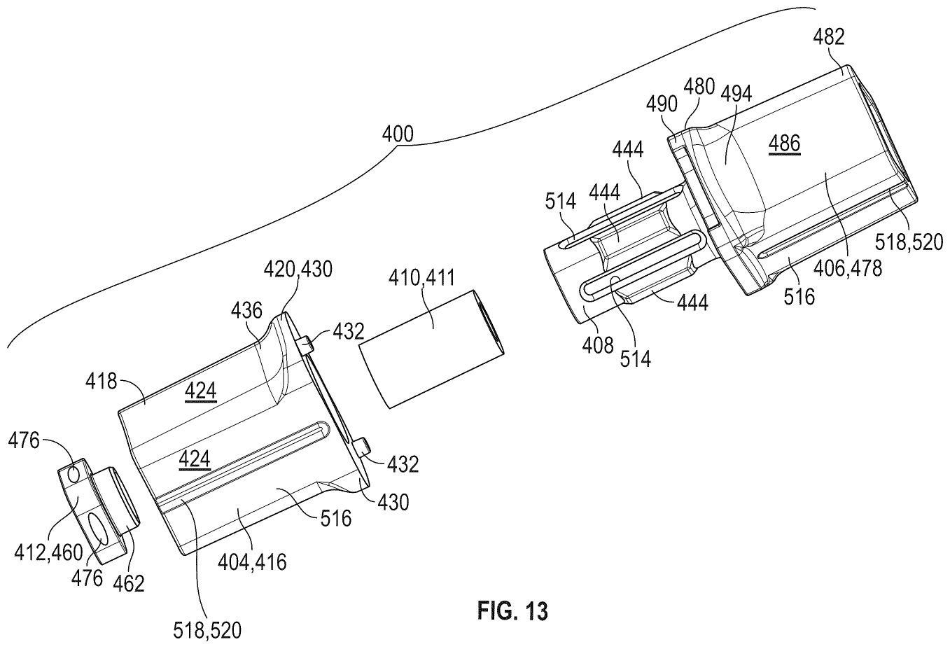

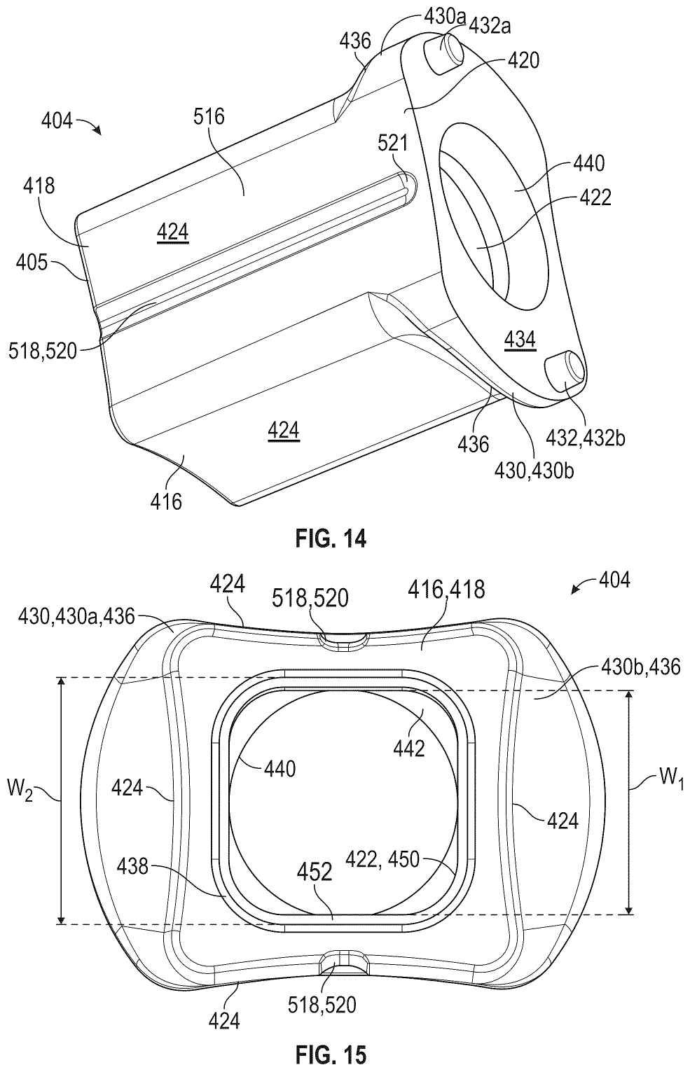

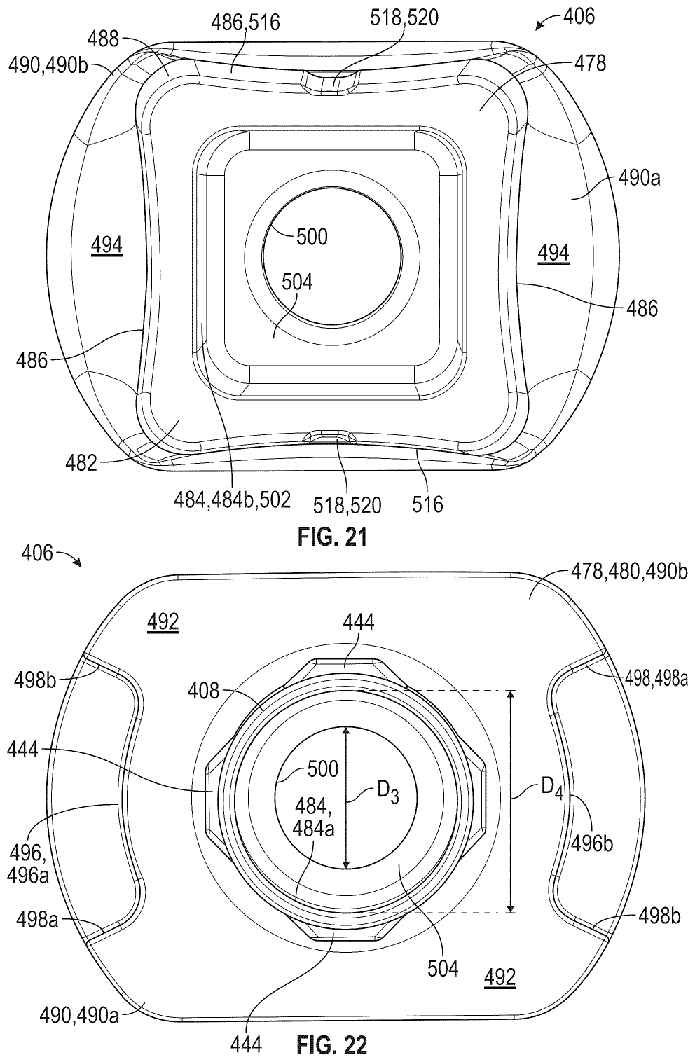

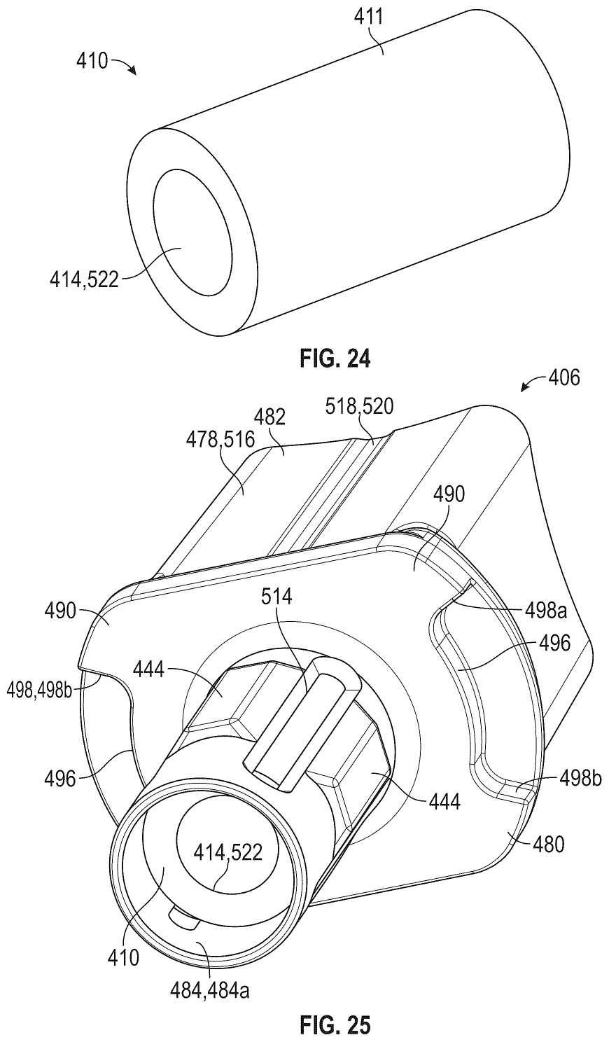

FIGS. 11-13 illustrate a representative example of the clamping device 400. As shown in FIG. 13, the clamping device 400 can comprise four main components: a first member 404, a second member 406 including an annular element 408, an elastomeric member 410, and a cap member 412. In some examples, the cap member 412 and/or the elastomeric member 410 can be optional. When the clamping device 400 is assembled, the first and second members 404, 406 can be rotatably coupled to one another and can be axially fixed relative to one another. The first and second members 404, 406 can each define an inner bore 422 (FIG. 14) and 484 (FIG. 21) extending longitudinally through the first and second members, respectively, which together form an inner bore 402 of the clamping device 400. The elastomeric member 410 can be a tubular member having a main body 411 defining a second inner bore 414 (FIG. 24) through which a shaft of the delivery catheter, such as shaft 102, can extend. The elastomeric member 410 can be disposed within the annular element 408 of the second member 406, as described further below.

The first and second member 404, 406 can be sized to allow a user to easily grip and rotate the first and second members 404, 406 relative to one another. For example, the overall length of the clamping device 400 can be between about 1 inch and about 2 inches, for instance between about 1.2 inches and about 1.5 inches. The length of the clamping device 400 can advantageously facilitate use, as the length of the first and second members provides additional leverage for locking/clamping and releasing the device. Further, the outer surfaces 424, 486 of the first and second members 404, 406 can be sized and shaped to accommodate ergonomic gripping by a user. For instance, the outer surfaces 424, 486 can be concave surfaces which are ergonomically configured (e.g., sized and shaped) to accommodate a user's fingers. Still further, the illustrated examples avoid ridges or shoulders on the outer surface of the clamping device, which mitigates discomfort to the user and provides a smooth, even surface for gripping.

Referring now to FIGS. 14-16, the first member 404 can comprise a main body 416 having a first end portion 418, a second end portion 420, and an inner lumen or bore 422 extending through the length of the main body 416. The main body 416 can have a rectangular prism shape with rounded corners and including a plurality of outer surfaces 424. As can be seen in FIG. 15, one or more of the outer surfaces 424 can be configured as concave surfaces, which advantageously allow a user to more easily grasp the first member 404 in order to, for example, rotate the first member 404 relative to the second member 406.

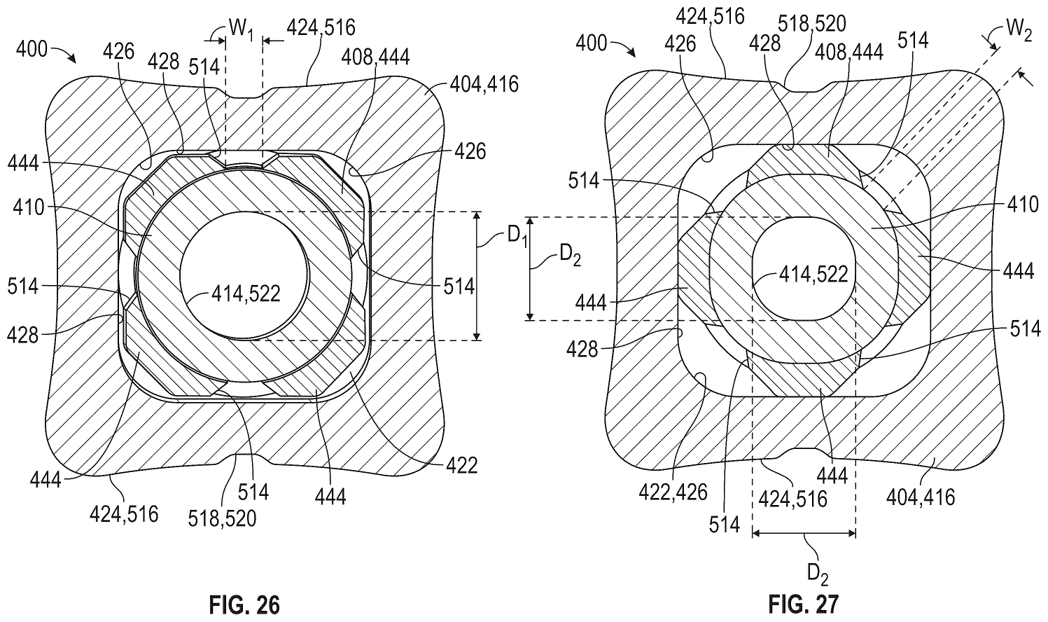

Referring still to FIG. 15, the inner bore 422 can have a non-circular cross-section having a shape that generally corresponds to the shape of the non-circular cross-section of the annular clement 408 when the bore 422 and the annular element 408 are rotationally aligned, as described in more detail below. In the illustrated example, as can be seen in the cross-sectional view shown in FIG. 26, the cross-sectional profile of the bore 422 (taken in a plane perpendicular to the longitudinal axis of the first member 404 and the bore 422; e.g., along line A-A in FIG. 11) comprises a substantially square shape including rounded corner portions 426 and flat side portions 428. However, in other examples, the annular clement 408 and the bore 422 can have any of various other corresponding non-circular shapes in cross-section including, but not limited to, elliptical, triangular, rectangular, cruciform (cross-shaped), flat-oval shaped (e.g., a shape comprising an oval with flat sides), etc.

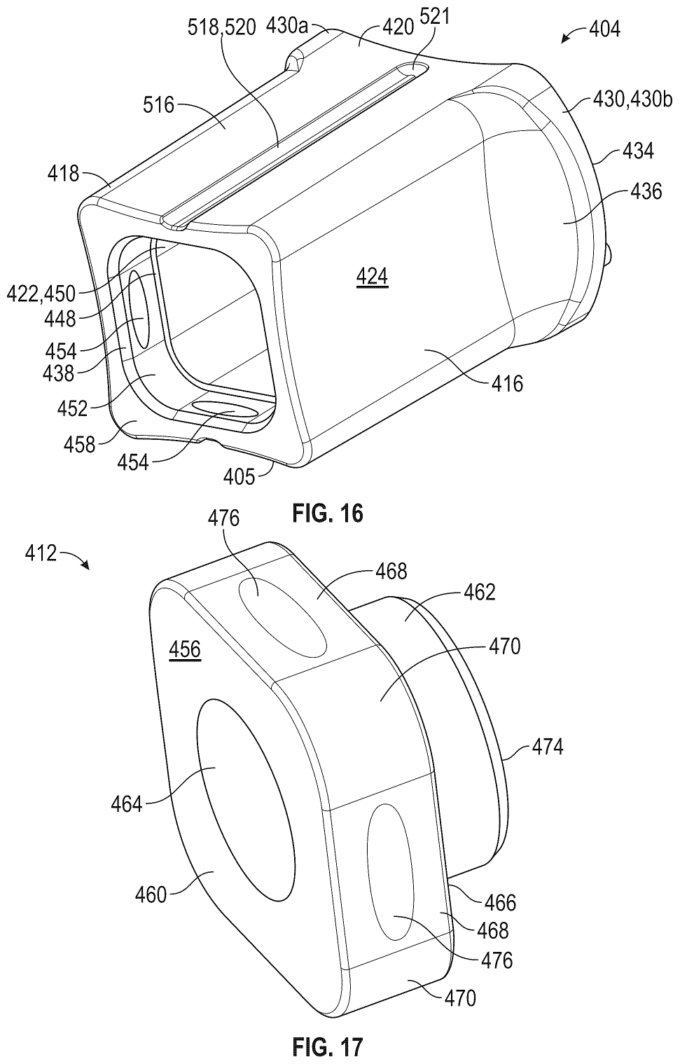

Referring to FIG. 14, the second end portion 420 of the first member 404 can comprise one or more flared portions/flanges/tabs 430 that extend laterally from the main body 416 in a direction away from the inner bore 422. Each flange 430 can comprise a protrusion 432, such as a cylindrical protrusion, extending axially from a first surface 434 of the flange. In the illustrated example, the second end portion 420 comprises a first flange 430a comprising a first protrusion 432a and a second flange 430b comprising a second protrusion 432b. The protrusions 432 can be disposed such that they are diametrically opposite one another relative to a longitudinal axis extending through the inner bore 422. As shown in FIGS. 14 and 16 The opposing second surface 436 of each flange 430 can be configured as a curved or ramped surface. Such a configuration allows a user to firmly grip the first member 404 such that their fingers abut the second surface 436 in a comfortable and ergonomic manner.

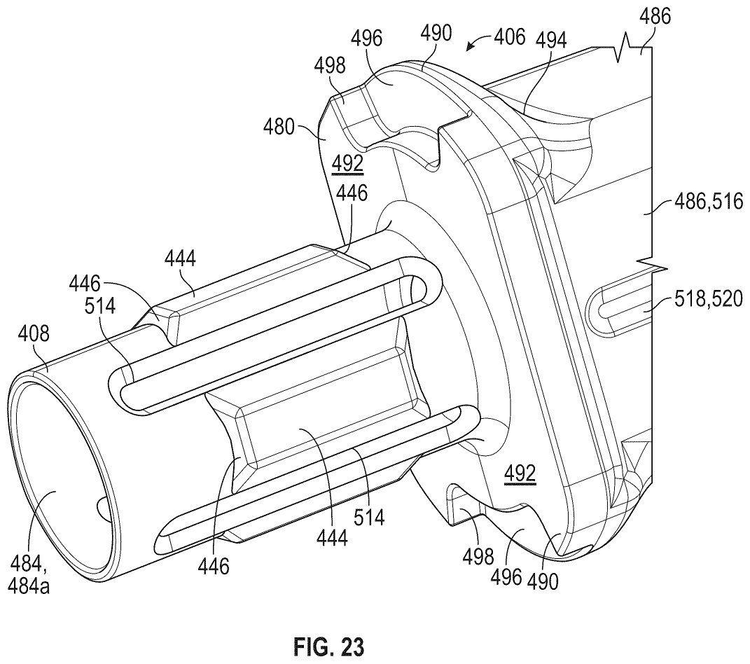

The first end portion 418 can define a first aperture or opening 438 and the second end portion 420 can define a second aperture or opening 440. In some instances, the first aperture 438 can have a non-circular shape and the second aperture 440 can have a circular shape. The first and/or second apertures 438, 440 can optionally comprise chamfered edges. As shown in FIG. 15, second end portion 420 can comprise a shoulder 442 extending toward a longitudinal axis of the first member 404 and defining the second opening 440. When the first and second members 404, 406 are coupled together, the shoulder 442 can engage a second end surface 446 (FIG. 23) of engagement members 444 of the second member's 406 annular element 408 to retain the first and second members 404, 406 axially relative to one another, as described in more detail below. Referring to FIG. 16, the inner bore 422 can comprise a shoulder/lip/ledge 448. A first end portion 450 of the inner bore can extend from the ledge 448 to the shoulder 442, and a second end portion 452 can extend from the ledge 448 to the first aperture 438. As shown in FIG. 15, the first end portion 450 can have a first width W₁ less than a second width W₂ of the second portion 452.

As can be seen in FIG. 16, the second end portion 452 of the inner bore 422 can comprise one or more recesses 454 having any of various shapes. For instance, in the illustrated example, the second end portion 452 can comprise four recesses 454 each having an oval shape. The recesses are equally spaced about an internal perimeter of the second end portion 418, however, in other examples, the recesses 454 can be disposed with unequal spacing and/or such that two or more recesses are disposed on the same side, etc.

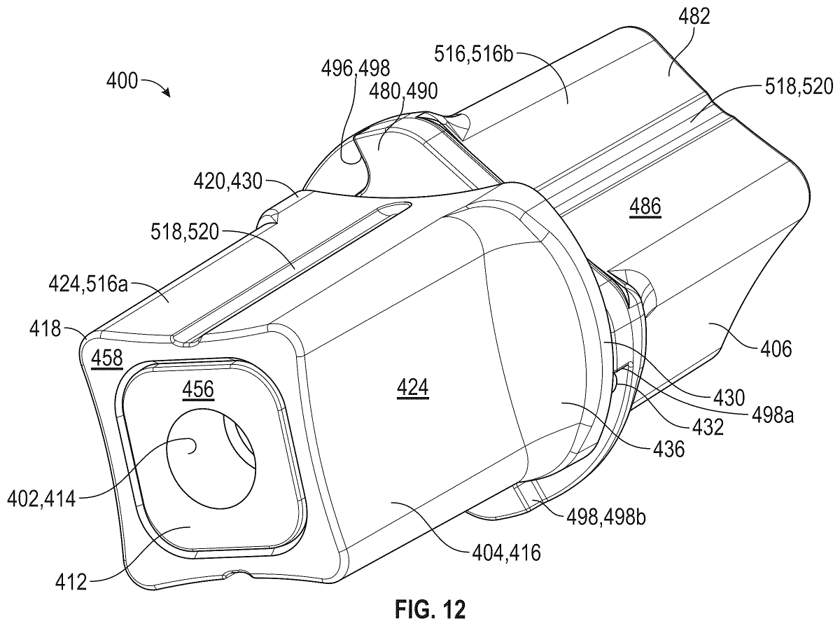

Referring to FIGS. 17-19, the clamping device 400 can further comprise a plug or cap member 412 configured to retain the elastomeric member 410 within the inner bore 422 of the first member 404. In some examples, such as the example illustrated in FIGS. 17-19, the cap member 412 can be an inset cap member that can be disposed within the second end portion 452 of the inner bore 422 (such as in a snap-fit configuration) such that a first surface 456 of the cap member 412 is flush with a first end surface 458 of the first member 404, as shown in FIG. 12. However, in other examples, such as shown in FIGS. 30-31, the cap member can be configured to be disposed at least partially over the first end portion 418 of the first member 404.

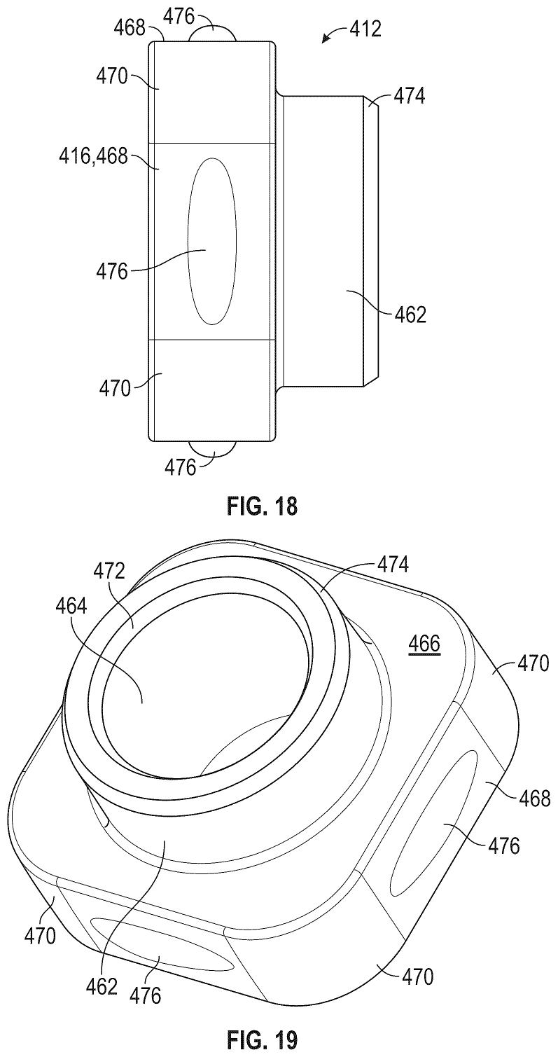

The cap member 412 can comprise a main body 460, an annular projection 462, and an inner bore 464 extending through the main body 460 and the annular projection 462. The main body 460 can have a first surface 456, mentioned previously, a second surface 466, and multiple side surfaces 468 connected to one another by curved corner portions 470. In some examples, as shown in FIGS. 17-19, the annular projection 462 can comprise chamfered inner and outer lips 472 (FIG. 19), 474. The inner bore 464 of the cap member 412 can have a circular shape in cross section and can be sized such that catheter shaft 102 can pass through the inner bore. The outer perimeter of the main body 416 can correspond to the first aperture 438. Referring to FIG. 18, the side surfaces 468 of the main body 416 can comprise a plurality of protrusions 476 extending from the side surfaces 468 in a direction away from the inner bore 464. The protrusions 476 can be sized and shaped to correspond with the recesses 454 of the first member 404 such that when the cap member 412 is disposed within the second portion 452 of the inner bore 422 the protrusions 476 sit within the recesses 454 and form a snap-fit connection between the cap 412 and the first member 404. This snap-fit configuration can advantageously case assembly, reduce manufacturing costs, and avoid higher costs manufacturing techniques such as ultrasonic welding.

Referring to FIG. 29, the cap member 412 can be inserted into the second aperture 438 such that the annular projection 462 is disposed in the first end portion 450 of the inner bore 422 and the main body 460 is disposed in the second end portion 452 of the inner bore (with the protrusions 476 disposed within corresponding recesses 454). The second surface 466 of the main body 460 can engage the ledge 448. As mentioned previously, and as shown in FIG. 12, when the cap member 412 is disposed within the first member 404 in a snap-fit connection, the first surface 456 of the cap member 412 is flush with the first end surface 458 of the first member. In other examples, the cap member 412 can be coupled to the first member 404 in any of various other ways, such as by thermal bonding, overmolding, adhesives, mechanical means such as screws or pins, and/or other means of coupling.

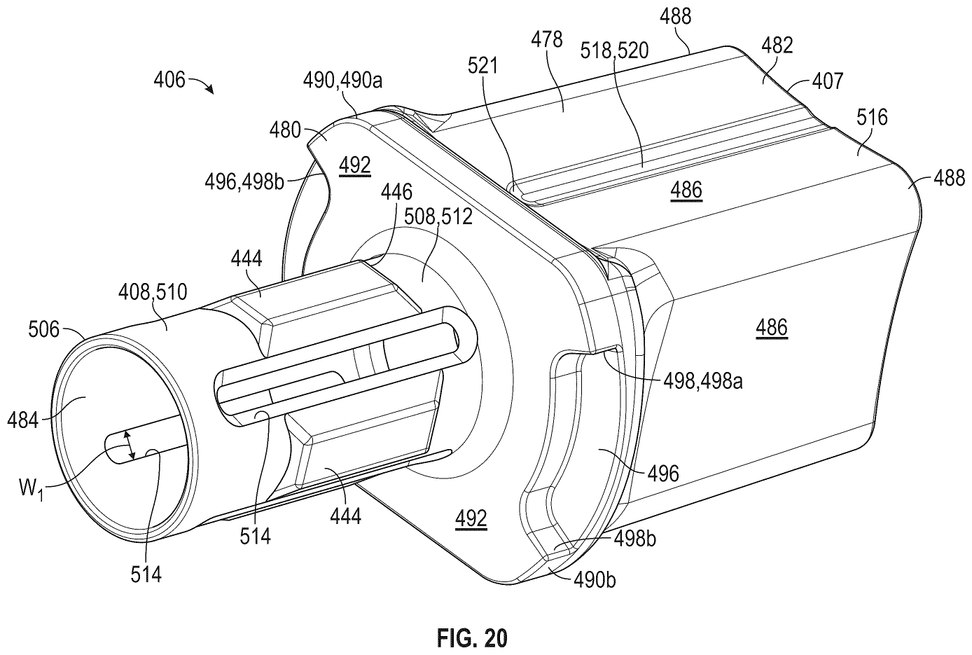

Referring to FIGS. 20-23, the second member 406 can comprise a main body 478 having a first end portion 480 and a second end portion 482, an annular element 408 extending axially from the first end portion 480 in a direction away from the second end portion 482, and an inner lumen or bore 484 (FIG. 21) extending longitudinally through the main body 478 and annular element 408. In some examples, such as the illustrated example, the annular clement 408 can be formed integrally with the main body 478, such as during the molding process. In other examples, the annular element 408 can be formed separately and coupled to the body 478 in various other ways, such as by snap-fit, thermal bonding, overmolding, adhesive, mechanical means such as screws, and/or other means of coupling.

The main body 478 can have a rectangular prism shape including a plurality of outer surfaces 486 coupled to eachother via curved corner portions 488. As can be seen in FIG. 21, one or more of the outer surfaces 486 can be configured as concave surfaces, which advantageously allow a user to more easily grasp the second member 406 in order to, for example, rotate the second member 406 relative to the first member 404, or vice versa.

Referring to FIG. 20, the first end portion 480 can comprise one or more flared portions/flanges/tabs 490 that extend from the main body 478 in a direction away from a longitudinal axis of the main body 478. The flanges 490 can comprise a first surface 492 (which is also the first end surface of the main body 478) oriented toward the first member 404 when the clamping device 400 is assembled, and an opposing second surface 494 (FIG. 21). Referring to FIG. 23, the second surface 494 of each flange 490 can be configured as a curved or ramped surface. Such a configuration allows a user to firmly grip and actuate the second member 406 such that their fingers abut the second surface 494 in a comfortable and ergonomic manner.

The first surface 492 of each flange 490 can comprise a recessed portion 496 disposed at a radially outer edge of the flange 490. In the illustrated example, the second member 406 comprises first and second flanges 490a, 490b each of which comprises a recessed portion 496a, 496b. The recessed portions 496 can have an arcuate shape that corresponds to the outer edge of the flanges 490. The recessed portions 490 can have a thickness and a width configured to accommodate the insertion of the protrusions 432a, 432b of the first member 404 within respective recesses 496a, 496b when the first and second members 404, 406 are coupled together, such as shown in FIG. 11.

Each recess 496 can comprise one or more stopping surfaces 498 configured to abut the protrusions 432 and prevent advancement of the protrusions 432 past a selected point. For instance, when the first and second members 404, 406 are rotated relative to one another, the protrusions 432a, 432b can slide within their respective recesses 496a, 496b until they abut the stopping surfaces 498. Such a configuration advantageously prevents the clamping device 400 from being over-tightened by providing a “hard stop” for both the release state and clamped/locked state positions. For instance, when the clamping device 400 is in the release state, the protrusions 432 can abut a first stopping surface 498a, and when the clamping device 400 is in the clamped state the protrusions 432 can abut a second stopping surface 498b. The first stopping surfaces 498a can be diametrically opposite one another, and the second stopping surfaces 498b can be diametrically opposite one another.

Referring to FIG. 21, first end portion 480 (FIG. 20) of the second member 406 can define a first aperture or opening 500 and the second end portion 482 can define a second aperture or opening 502. In some instances, such as the illustrated example, the first aperture 500 can have a circular shape and the second aperture 502 can have a non-circular shape. As shown in FIG. 22, the first aperture 500 can have a diameter D₃ smaller than the diameter D₄ of the inner bore 484 of the annular element 408 such that a shoulder 504 is defined between the inner bore 484a of the annular element 408 and the inner bore 484b of the main body 478. The shoulder 504 can help retain the elastomeric member 410 within the annular element 408.

Referring to FIG. 20, the annular element 408 can have a first, free end portion 506 and a second end portion 508 coupled to the first end portion 480 of the main body 478. The annular element 408 can comprise one or more engagement members 444 (e.g., four in the illustrated example). The engagement members 444 can extend longitudinally along the annular element 408 from a first cylindrical portion 510 to a second cylindrical portion 512 of the annular element, and can be spaced apart from each other about a circumference of the annular element 408, as shown in FIG. 22. Referring to FIG. 22, the engagement members 444 can protrude radially outward from outer surfaces of the cylindrical portions 510, 512 to define a non-circular cross-sectional profile of the annular element 408 (the cross-sectional profile being taken perpendicular to the longitudinal axis of the annular element). When the clamping device 400 is in the clamped state, the engagement members 444 can deflect inwardly toward the inner bore 484a. The engagement members 444 can each have first and second longitudinal end surfaces 446, one of which is configured to abut the shoulder 442 of the first member 404 such that the first and second members 404, 406 form a snap-fit connection whereby they can rotate relative to one another but are restrained against axial movement relative to one another. This snap-fit configuration can advantageously case assembly, reduce manufacturing costs, and avoid higher costs manufacturing techniques such as ultrasonic welding.

In the illustrated example, the engagement members 444 are chamfered rectangular protrusions. However, in other examples, the engagement members 444 can have other shapes, including but not limited to cuboid, elliptical, ovular, triangular, etc. Such shapes can also comprise chamfered portions. Though the illustrated example shows four engagement members 444, in other examples, the annular element 408 can comprise a greater or fewer number of engagement members 444. For example, in some examples, the annular element can comprise one, two, three, or five engagement members.

The annular element 408 can further comprise one or more slots or openings 514 (e.g., four in the illustrated example) extending through a thickness of the annular element 408 and extending longitudinally along less than a full length of the annular element 408. In some instances, such as the illustrated example, the openings 514 can have an elongated square oval shape including two rounded end portions. The openings 514 can be spaced apart about the circumference of the annular element 408, for example, between adjacent engagement members 444. The openings 514 allow the annular element 408 to deflect radially inwardly when a compressive force is applied to an outer surface of the annular element 408. The one or more openings 514 can have a first width W₁ when the clamping device 400 is in the release state (see FIG. 26), and a second width W₂ when the clamping device 400 is in the clamped state (see FIG. 27). In the illustrated example, the first width W₁ is the same for each opening 514 and the second width W₂ is the same for each opening 514, however, in other examples, the widths may vary between openings. For instance, one or more of the openings 514 could have a narrower or wider first width W₁ and/or second width W₂ relative to the other openings 514.

Though the illustrated example shows four openings 514, in other examples, the annular element can comprise a greater or fewer number of openings. For example, in some examples, the annular element can comprise one, two, three, or five openings. In some instances, the edges surrounding the openings 514 can be chamfered, such as shown in the illustrated example.

Referring again to FIG. 12, the first and second members 404, 406 can each comprise one or more surfaces 424, 486 configured as indicator surfaces 516. The indicator surfaces 516 can indicate to a user whether the clamping device 400 is in the clamped/locked state or the release state. For instance, the first member 404 can comprise a first indicator surface 516a, and the second member 406 can comprise a second indicator surface 516b. When the indicator surfaces 516a, 516b are rotationally aligned (see FIG. 28), the clamping device 400 is in the clamped/locked state. As used herein, the term “rotationally aligned” means that the first indicator surface 516a is in a first rotational position relative to the second indicator surface 516b such that the surfaces are parallel to one another, such as shown in FIG. 28. When the indicator surfaces 516a, 516b are rotationally offset from one another (see FIGS. 11-12), the clamping device 400 is in the release state. As used herein, the term “rotationally offset” means that the first indicator surface 516a is in a rotational position relative to the second indicator surface 516b such that the two surfaces 516 are at an angle relative to one another. In other examples, rotational offset of the indicator surfaces 516 can indicate that the clamping device 400 is in the locked state and rotational alignment of the indicator surfaces 516 can indicate that the clamping device is in the release state.

In some instances, the indicator surfaces 516 can additionally comprise indicia 518 configured to further indicate to a user whether the clamping device 400 is in the clamped/locked state or the release state. The indicia 518 can be, for example, tactile indicia (such as a surface texture, a raised marking, a recessed marking, a cutout, a different material, etc.) and/or visual indicia (such as a line or symbol or colored section, etc.). In the illustrated example, the indicia 518 are recessed troughs/depressions/channels 520 extending at least partially along the length of the respective indicator surfaces 516. The channels 520 can each extend from a respective edge 405, 407 (FIG. 11) of the first and second members 404, 406 and along a length of the first and second members, terminating in a rounded end portion 521. In some instances, such as shown in FIGS. 30-31, one or more of the channels 520 can comprise more than one rounded end portion 521.

In use, a physician can feel the channels 520 and thus determine whether the clamping device 400 is in the clamped or released state without needing to look at the device. Further, the channels 520 can facilitate a physician's grip on the clamping device by providing a textured surface. In the illustrated example, the clamping device 400 is in the clamped state when the indicia 518 are aligned with one another along the length of the clamping device, as shown in FIG. 28. Though the channels 520 are already visible to a physician, the indicia 518 can in some examples comprise a further visual element, such as a stripe of colored paint disposed along the length of the channel 520.

The indicia 518 can be formed integrally with the first and second members 404, 406, for example, during the molding process. However, in other examples, the indicia 518 can be formed separately and bonded with the first and second members, such as by thermal bonding, adhesive, laser welding, overmolding, and/or mechanical means such as screws, etc

FIG. 24 illustrates one example of the elastomeric member 410. When the clamping device 400 is in the clamped state, the elastomeric member 410 can frictionally engage the outer surface of a shaft extending through the clamping device, to restrain the clamping device against movement relative to the shaft. In the illustrated example, the elastomeric member 410 is a cylindrical, tubular member 411. However, in other examples, the elastomeric member 410 can take any of various forms having an outer profile corresponding to the inner profile of the annular element 408. The main body 411 of the elastomeric member 410 can define a lumen 414 extending along the length of the elastomeric member. The lumen 414 can be sized to receive the shaft of a medical instrument, for example, a delivery catheter. FIG. 25 shows the elastomeric member disposed within the annular element 408.

Referring to FIGS. 26-27, which show a transverse cross-section of the clamping device 400 taken along line A-A with the elastomeric member 410 disposed within the annular element 408, when the clamping device 400 is in the release state (FIG. 26) the lumen 414 can have a first diameter D₁ such that the clamping device 400 can be moved along the length of the catheter shaft, and when the clamping device is in the clamped state (FIG. 27) the lumen 414 can have a second diameter D₂ smaller than the first diameter. In some cases, such as shown in FIG. 27, the lumen 414 can deform into a non-circular shape having a width the same size as the second diameter D₂. The second diameter D₂ can be sized to frictionally engage the surface of the catheter shaft such that the clamping device 400 and the shaft are restrained from movement relative to one another. As shown in FIG. 26, the outer diameter of the elastomeric member 410 can be slightly smaller than the inner diameter of the annular element 408 such that the elastomeric member 410 is retained within the annular clement 408. As shown in FIG. 29, the elastomeric member 410 can be restrained from sliding out of the annular element 408 at a first end by the cap 412, and at a second end by the shoulder 504 of the second member 406.

The elastomeric member 410 can comprise natural rubber, any of various synthetic elastomers, such as silicone rubber or polyurethane, or various combinations thereof. In some implementations, the elastomeric member can comprise thermoplastic polyurethane (TPU), such as NEUSoft™ UR852A (NEU Specialty Engineered Materials). The inner surface 522 of the elastomeric member 410 can be tacky/sticky/adhesive such that it provides additional friction against the outer surface of the catheter shaft when the clamping device 400 is in the clamped state, which can advantageously provide two or more times the locking force as would be provided by an elastomeric member comprising a non-sticky material.

The clamping device 400 can be assembled by disposing the elastomeric member 410 within the annular element 408 and inserting the annular element 408 through the second aperture 440 into the first end portion 450 of the inner bore 422 of the first member 404 to form a snap-fit connection between the first and second members 404, 406. As the annular element 408 is inserted into the bore 422, the shoulder 442 of the first member 404 can deflect the engagement members 444 radially inward, compressing the annular element 408 (and therefore the elastomeric member 410) to a reduced diameter such that the annular element 408 can pass through the second aperture 440. Once the engagement members 444 have passed through the second aperture 438, the annular element 408 can return to its uncompressed diameter within the first portion 450 of the inner bore 422. As the uncompressed external diameter of the annular element 408 (including engagement members 444) is greater than the diameter of second aperture 438, the annular element 408 is retained within the inner bore 422. The end surfaces 446 (see e.g., FIG. 23) of the engagement members 444 can abut the shoulder 442, restraining the first and second members 404, 406 from axial movement relative to one another. The cap 412 can be inserted into the first end portion 452 of the inner bore 422 of the first member 404 to retain the elastomeric member 410 within the annular element 408.

Thus assembled, the first member 404 and the second member 406 are fixed axially relative to one another and can be rotated relative to one another to move the clamping device 400 between the release state and the clamped state.

The first and second members 404, 406 can be rotated relative to one another by applying opposing rotational forces to the first and second members 404, 406. For instance, a physician can grip the first and second members 404, 406 (e.g., one in each hand) and twist the first member 404 while maintaining the second member 406 in a fixed position, or vice versa. The first and second members 404, 406 can be rotated relative to one another until the protrusion 432 contacts the either stopping surface 498, depending on whether the clamping device is being moved into the release state or the clamped state. This configuration allows a physician to quickly and easily move the clamping device between the release state and the clamped state, and also to easily see whether the clamping device 400 is in the released state or the clamped state based on the position of the indicator surfaces 516 and/or indicia 518 relative to one another.

Referring to FIG. 26, when the clamping device 400 is in the release state, the inner bore 422 of the first member 404 and the outer surface of the annular element 408 are rotationally aligned. As used herein, the term “rotationally aligned” means that the annular element 408 is in a first rotational position relative to the bore 422 such that the cross-sectional profile of the annular element corresponds to the cross-sectional profile of the bore 422. In other words, the annular element 408 (and therefore the elastomeric member 410) is not compressed or deflected by the bore 422. In the illustrated example, when the bore 422 and the annular element 410 are rotationally aligned, the engagement members 444 of the annular element 408 extend into the rounded corner portions 426 of the bore 422 and the openings 514 in the annular element 408 have a first width W₁. In the rotationally aligned state, the lumen 414 of the elastomeric member 410 has a first diameter D₁ sized such that the clamping device 400 is movable relative to a shaft extending through the inner bore 402.

Referring now to FIG. 27, when the clamping device 400 is in the clamped state, the inner bore 422 of the first member 404 and the outer surface of the annular element 408 are rotationally offset. As used herein, the term “rotationally offset” means that the annular clement 408 is in a rotational position relative to the bore 422 such that the cross-sectional profile of the annular element 408 does not correspond to the cross-sectional profile of the bore 422. In this position, the cross-sectional profile of the bore 422 exerts a compressing force on the annular clement 408 (and therefore on the elastomeric member 410). In the illustrated example, when the bore and the annular element are rotationally offset, the flat side portions 428 of the bore 422 contact the engagement members 444, deflecting the engagement members 444 radially inward toward the inner lumen 414 of the elastomeric member 410. As the engagement members 444 are deflected inwardly they move toward one another such that the openings 514 narrow or deform to a second width W₂ and such that the lumen 414 narrows or deforms to a second diameter (or width) D₂. In the rotationally offset state, the second diameter D₂ is sized such that the internal surface 522 of the elastomeric member 410 engages the outer surface of a shaft extending through the lumen 414. The frictional force of the elastomeric member 410 against the outer surface of the shaft is sufficient to resist longitudinal movement of the shaft relative to the clamping device 400, and vice versa.

In alternative examples, the clamping device 400 does not include an elastomeric member 410 and instead inner surface portions of the annular element 408 can clamp against and grip the outer surface of the shaft. For example, the annular element 408 can be sized such that the inner surfaces of the engagement members 444 come into contact with the outer surface of the shaft when clamping device 400 is in the clamped state. In some examples, a thin layer of an elastomeric material (e.g., silicone rubber or any of the materials described previously as forming the elastomeric member) can be secured, such as with an adhesive, to the inner surface of each engagement member 444, and the layers of elastomeric material can come into contact with the outer surface of the shaft when clamping device 400 is in the clamped state.

The clamping device 400 can be used to restrict movement of a shaft of a first medical device relative to another medical device through the shaft extends. For example, while the clamping device 400 is in the release state, a physician can slide the clamping device 400 along the shaft of the first medical device until the clamping device reaches a selected clamping site. In some examples, for example, the selected clamping site can be adjacent the proximal end portion of a second medical device (e.g., an introducer assembly) through which the first medical device extends.

Once the clamping device 400 is positioned at the selected clamping site, the physician can actuate the clamping device 400 to move the device from the release state to the clamped state. For example, the physician can hold the first member 404 and rotate the second member 406 relative to the first member, or vice versa. As the annular element 408 rotates into the rotationally offset position relative to the inner bore 422 of the first member 404, the annular element 408 (and therefore the elastomeric member 410) is compressed by the bore 422, thereby reducing the lumen 414 of the elastomeric element 410 from the first diameter D₁ to the second diameter D₂. When compressed to the second diameter D₂, the inner surface of the elastomeric clement 410 frictionally engages the outer surface of the shaft, preventing the clamping device 400 from moving relative to the shaft. Thusly clamped to the shaft, the clamping device 400 abuts the proximal end of the second medical device and prevents distal movement of the first shaft relative to the second medical device.

The first medical device can comprise any medical device comprising a shaft, including, but not limited to, a catheter (e.g., catheter 100), a cannula, a needle or a medical probe. The second medical device can be any medical device that has an opening or bore sized to receive the shaft of the first medical device.

In one specific implementation, as depicted in FIG. 10, the first medical device is a catheter 100 having a shaft 102 and the second medical device is an introducer assembly 300.

In another implementation, the first medical device is a first catheter having a first shaft and the second medical device is a second catheter having a second shaft. The first shaft extends through the second shaft. When clamped onto the first shaft, the clamping device can abut the proximal end of the second catheter and prevent further movement of the first catheter relative to the second catheter.

FIGS. 30-31 illustrate another example of a clamping device 400 having a cap 524 in lieu of or in addition to cap 412. The cap 524 can be a permanent or removable member that extends over the first end portion 418 of the first member 404. In some instances, the cap 524 can form a snap-fit connection with the first member 404, in other instances, the cap 524 coupled to the first member 404 in any of various other ways, such as by thermal bonding, overmolding, adhesives, mechanical means such as screws or pins, and/or other means of coupling.

The cap 524 can comprise a main body 526 defining an inner bore 528 and having a substantially cylindrical shape and including one or more flat surface portions 530. The flat surface portions 530 can be aligned with the one or more indicator surfaces 516 of the first and second members 404, 406. The cap 524 can further comprise an annular element 532 extending axially from a first surface 534 of the cap and defining an opening 536 connected to the inner bore 528. The opening 536 can be sized such that catheter shaft 102 can pass through the opening 536 and into the inner bore 528. In some instances, such as the illustrated example, the annular element 532 can comprise one or more chamfered edge portions 538.

In some examples, such as the example shown in FIGS. 30-31, in lieu of a curved second surface 436, 494, the second surface 436, 494 of each flange 430, 490 can extend perpendicularly relative to an outer surface 424, 486 of the first and/or second members 404, 406, respectively.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

### Example 1

A clamping device for a shaft of a catheter, comprising:
a first member comprising a first main body having a first indicator surface, the first main body defining a first inner bore having a non-circular cross-sectional profile;
a second member comprising a second main body having a second indicator surface and an annular element extending from a first end portion of the main body and defining a second inner bore, the annular element received within the first inner bore and comprising an outer surface having a non-circular cross-sectional profile;
wherein the clamping device is movable between a first, release state in which the second member is in a first rotational position relative to the first member and the second inner bore has a first diameter, and a second, clamped state in which the second member is in a second rotational position relative to the first member and the second inner bore has a second diameter less than the first diameter; and
wherein when the clamping device is in the clamped state the first and second indicator surfaces are rotationally aligned with one another, and wherein when the clamping device is in the release state the first and second indicator surfaces are rotationally offset from one another.

### Example 2

The clamping device of any example herein, particularly example 1, further comprising an elastomeric member disposed within the annular element, the elastomeric member defining an inner lumen sized to receive a catheter shaft.

### Example 3

The clamping device of any example herein, particularly any one of examples 1-2, wherein when the clamping device is in the clamped state the clamping device is configured to engage an outer surface of a catheter shaft extending through the inner lumen, and wherein when the clamping device is in the release state the clamping device can be moved axially along a length of the catheter shaft.

### Example 4

The clamping device of any example herein, particularly any one of examples 1-3, wherein when the clamping device is in the clamped state the clamping device is restrained from axial movement relative to a catheter shaft extending through the second bore.

### Example 5

The clamping device of any example herein, particularly any one of examples 1-4, wherein the first and second indicator surfaces each comprise a respective channel extending at least partially along a length of the indicator surface.

### Example 6

The clamping device of any example herein, particularly any example 5, wherein when the clamping device is in the release state the channels are visually offset from one another.

### Example 7

The clamping device of any example herein, particularly any one of examples 5-6. wherein when the clamping device is in clamped state the channels are visually aligned with one another.

### Example 8

The clamping device of any example herein, particularly any one of examples 1-7, wherein the first and second indicator surfaces are concave.

### Example 9

The clamping device of any example herein, particularly any one of examples 1-8, wherein the first member comprises a third indicator surface opposite the first indicator surface and the second member comprises a fourth indicator surface opposite the second indicator surface.

### Example 10

The clamping device of any example herein, particularly any one of examples 1-9, wherein the first and second member comprise a plurality of outer surfaces and wherein the outer surfaces are concave.

### Example 11

The clamping device of any example herein, particularly any one of examples 1-10, wherein a user can ergonomically grip and actuate the clamping device using the indicator surfaces.

### Example 12

The clamping device of any example herein, particularly any one of examples 1-11, further comprising a cap member coupled to the first member.

### Example 13

The clamping device of any example herein, particularly example 12, wherein the cap member disposed within a first end portion of the first member.

### Example 14

The clamping device of any example herein, particularly example 13, wherein the cap member comprises a main body and an annular projection extending from the main body.

### Example 15

The clamping device of any example herein, particularly example 14, wherein the cap member further comprises a plurality of protrusions extending from side surfaces of the main body.

### Example 16

The clamping device of any example herein, particularly example 15, wherein a first end portion of the first member comprises a plurality of recesses corresponding to the plurality of protrusions such that the cap member forms a snap-fit connection inside the first end portion of the first member.

### Example 17

The clamping device of any example herein, particularly example 12, wherein the cap member is disposed over a first end portion of the first member.

### Example 18

The clamping device of any example herein, particularly any one of examples 1-17, wherein when in the clamped state, the first inner bore and the annular element are rotationally offset from one another such that the annular element is compressed by the first inner bore, thereby reducing the diameter of the second inner bore.

### Example 19

The clamping device of any example herein, particularly any one of examples 1-19, wherein the annular element comprises one or more engagement members extending longitudinally along the annular element and defining the non-circular outer profile of the annular element.

### Example 20

The clamping device of any example herein, particularly example 19, wherein the engagement members comprise chamfered rectangular protrusions.

### Example 21

The clamping device of any example herein, particularly any one of examples 19-20, wherein when the first inner bore and the annular element are rotationally offset from one another, the engagement members are deflected inwardly toward the second inner bore.

### Example 22

The clamping device of any example herein, particularly any one of examples 1-21, wherein when in the release state, the first inner bore and the annular element are rotationally aligned with one another.

### Example 23

The clamping device of any example herein, particularly any one of examples 1-22, wherein the first inner bore comprises a shoulder configured to engage one or more end surfaces of the one or more engagement members such that the first and second members form a snap-fit connection and are fixed axially relative to one another.

### Example 24

The clamping device of any example herein, particularly any one of examples 1-23, wherein the annular element comprises one or more openings extending longitudinally along the annular element and spaced apart about a circumference of the annular element.

### Example 25

The clamping device of any example herein, particularly example 24, wherein when the clamping device is in the release state the openings have a first width and when the clamping device is in the clamped state the openings have a second width narrower than the first width.

### Example 26

The clamping device of any example herein, particularly any one of examples 1-25, wherein the first member comprises one or more axially-extending protrusions disposed in one or more corresponding recesses of the second member.

### Example 27

The clamping device of any example herein, particularly example 26, wherein the recesses each comprise a first stopping surface and a second stopping surface and wherein when the first and second members are rotated relative to one another the one or more protrusions can abut the one or more stopping surfaces to prevent rotation of the first and second members relative to one another past a selected point.

### Example 28

The clamping device of any example herein, particularly example 27, wherein when the first and second members are in the release state the axially-extending protrusions abut the first stopping surfaces and when the first and second members are in the clamped state the axially-extending protrusions abut the second stopping surfaces.

### Example 29

The clamping device of any example herein, particularly any one of examples 1-28, wherein the first and second members each include a first end portion and a second end portion, and wherein each member comprises one or more flanges extending laterally from the second end portion of the first member and the first end portion of the second member, respectively.

### Example 30

The clamping device of any example herein, particularly example 29, wherein each flange comprises a first surface and a second opposing surface, and wherein the second surface is a ramped surface.

### Example 31

The clamping device of any example herein, particularly any one of examples 1-30, wherein the clamping device can be moved between the release state and the clamped state by rotating the first and second members relative to one another.

### Example 32

A catheter assembly, comprising:
a shaft; and
a clamping device disposed on the shaft, the clamping device comprising:
a first member comprising a first main body having a first indicator surface, the first main body defining a first inner bore having a non-circular cross-sectional profile, and
a second member comprising a second main body having a second indicator surface and an annular element extending from a first end portion of the main body, the annular element having a non-circular cross-sectional outer profile corresponding to the non-circular cross-sectional profile of the first inner bore, the second member defining a second inner bore extending through the annular element, the shaft extending through the second inner bore;
wherein the annular element is received within the first inner bore;
wherein the clamping device is movable between a first, release state in which the second member is in a first rotational position relative to the first member and the second inner bore has a first diameter, and a second, clamped state in which the second member is in a second rotational position relative to the first member and the second inner bore has a second diameter less than the first diameter; and
wherein when the clamping device is in the clamped state the first and second indicator surfaces are rotationally aligned with one another, and wherein when the clamping device is in the release state the first and second indicator surfaces are rotationally offset from one another.

### Example 33

The assembly of any example herein, particularly example 32, wherein when the second member is in the first rotational position the non-circular cross-section of the annular element is rotationally aligned with the non-circular cross-section of the first inner bore, and wherein when the second member is in the second rotational position the non-circular cross-section of the annular element is rotationally offset from the non-circular cross-section of the first inner bore.

### Example 34

The assembly of any example herein, particularly any one of examples 32-33, further comprising a tubular elastomeric member disposed within the annular element such that the shaft extends through a lumen of the elastomeric member.

### Example 35

The assembly of any example herein, particularly example 34, wherein when the clamping device is in the clamped state an inner surface of the elastomeric member frictionally engages the outer surface of the shaft.

### Example 36

The assembly of any example herein, particularly any one of examples 32-35, wherein the first and second indicator surfaces each comprise a respective channel extending at least partially along a length of the indicator surface.

### Example 37

The assembly of any example herein, particularly example 36, wherein when the clamping device is in the release state the channels are visually offset with one another.

### Example 38

The assembly of any example herein, particularly any one of examples 32-37, wherein when the clamping device is in clamped state the channels are visually aligned from one another.

### Example 39

The assembly of any example herein, particularly any one of examples 32-37, wherein the first and second indicator surfaces are concave.

### Example 40

The assembly of any example herein, particularly any one of examples 32-39, wherein a user can ergonomically grip and actuate the clamping device using the indicator surfaces.

### Example 41

The assembly of any example herein, particularly any one of examples 32-40, further comprising a cap member coupled to the first member.

### Example 42

The assembly of any example herein, particularly example 41, wherein the cap member disposed within a first end portion of the first member.

### Example 43

The assembly of any example herein, particularly example 41, wherein the cap member is disposed over a first end portion of the first member.

### Example 44

The assembly of any example herein, particularly any one of examples 32-43, wherein when in the clamped state, the first inner bore and the annular element are rotationally offset from one another such that the annular element is compressed by the first inner bore, thereby reducing the diameter of the second inner bore.

### Example 45

The assembly of any example herein, particularly any one of examples 32-44, wherein the annular element comprises one or more engagement members extending longitudinally along the annular element and defining the non-circular outer profile of the annular element.

### Example 46

The assembly of any example herein, particularly any one of examples 45, wherein when the first inner bore and the annular element are rotationally offset from one another, the engagement members are deflected inwardly toward the second inner bore.

### Example 47

The assembly of any one of claims 45-46, wherein the first inner bore comprises shoulder configured to engage one or more end surfaces of the one or more engagement members such that the first and second members form a snap-fit connection and are fixed axially relative to one another.

### Example 48

The assembly of any example herein, particularly any one of examples 32-47, wherein when in the release state, the first inner bore and the annular element are rotationally aligned with one another.

### Example 49

The assembly of any example herein, particularly any one of examples 32-48, wherein the annular element comprises one or more openings extending longitudinally along the annular element and spaced apart about a circumference of the annular element.

### Example 50

The assembly of any example herein, particularly any one of examples 32-49, wherein the first member comprises one or more axially-extending protrusions disposed in one or more corresponding recesses of the second member, and wherein the recesses comprise one or more stopping surfaces and wherein when the first and second members are rotated relative to one another the one or more protrusions can contact the one or more stopping surfaces to prevent rotation of the first and second members relative to one another past a selected point.

### Example 51

The clamping device of any example herein, particularly any one of examples 32-50, wherein the clamping device can be moved between the release state and the clamped state by rotating the first and second members relative to one another.

### Example 52

A clamping device, comprising:
a first member comprising a first main body having a first indicator surface and an axially-extending protrusion, the main body defining a first inner bore extending therethrough;
a second member comprising a second main body having a second indicator surface, a recess within which the axially-extending protrusion of the first member is disposed, and an annular element extending from a first end portion of the main body axially away from a second end portion of the main body and into the first inner bore, the annular element defining a second inner bore extending therethrough;
wherein the clamping device is movable between a first, release state in which the second inner bore has a first diameter, and a second, clamped state wherein the second inner bore has a second diameter smaller than the first diameter; and
wherein the recess comprises a first stopping surface and a second stopping surface and wherein when the first and second members are in the release state the axially-extending protrusion abuts the first stopping surface and when the first and second members are in the clamped state the axially-extending protrusion abuts the second stopping surface.

### Example 53

The clamping device of any example herein, particularly example 52, wherein the inner bore of the first member has a non-circular cross-sectional profile and wherein the annular element comprises an outer surface having a corresponding non-circular cross-sectional profile.

### Example 54

The clamping device of any example herein, particularly any one of examples 52-53, wherein when the second member is in a first rotational position relative to the first member the clamping device is in the release state, and wherein when the second member is in a second rotational position relative to the first member the clamping device is in the clamped state.

### Example 55

The clamping device of any example herein, particularly example 54, wherein when the second member is in the first rotational position the non-circular cross-section of the annular element is rotationally aligned with the non-circular cross-section of the inner bore of the first member, and wherein when the second member is in the second rotational position the non-circular cross-section of the annular element is rotationally offset from the non-circular cross-section of the inner bore of the first member.

### Example 56

The clamping device of any example herein, particularly any one of examples 52-55, further comprising an elastomeric member disposed within the annular element, the elastomeric member defining an inner lumen sized to receive a catheter shaft.

### Example 57

The clamping device of any example herein, particularly any one of examples 52-56, wherein when the clamping device is in the clamped state the clamping device is configured to engage an outer surface of a catheter shaft extending through the second inner bore, and wherein when the clamping device is in the release state the clamping device can be moved axially along a length of the catheter shaft.

### Example 58

The clamping device of any example herein, particularly any one of examples 52-57, wherein when the clamping device is in the clamped state the clamping device is restrained from axial movement relative to a catheter shaft extending through the second bore.

### Example 59

The clamping device of any example herein, particularly any one of examples 52-58, wherein the first and second indicator surfaces each comprise a respective channel extending at least partially along a length of the indicator surface.

### Example 60

The clamping device of any example herein, particularly example 59, wherein when the clamping device is in the release state the channels are visually offset from one another.

### Example 61

The clamping device of any example herein, particularly any one of examples 59-60, wherein when the clamping device is in clamped state the channels are visually aligned with one another.

### Example 62

The clamping device of any example herein, particularly any one of examples 52-61, wherein the first and second indicator surfaces are part of a plurality of outer surfaces of the first and second member and wherein the outer surfaces are concave.

### Example 63

The clamping device of any example herein, particularly example 62, wherein a user can ergonomically grip and actuate the clamping device using the concave outer surfaces.

### Example 64

The clamping device of any example herein, particularly any one of examples 52-63, further comprising a cap member coupled to the first member, the cap member comprising a main body and an annular projection extending from the main body.

### Example 65

The clamping device of any example herein, particularly example 64, wherein the cap member disposed within a first end portion of the first member in a snap-fit connection.

### Example 66

The clamping device of any example herein, particularly example 64, wherein the cap member is disposed over a first end portion of the first member.

### Example 67

The clamping device of any example herein, particularly any one of examples 52-66, wherein when in the clamped state, the first inner bore and the annular element are rotationally offset from one another such that the annular element is compressed by the first inner bore, thereby reducing the diameter of the second inner bore.

### Example 68

The clamping device of any example herein, particularly any one of examples 52-67, wherein the clamping device can be moved between the release state and the clamped state by rotating the first and second members relative to one another.

### Example 69

A catheter assembly, comprising:
a shaft; and
a clamping device disposed on the shaft, comprising:
a first member comprising a first main body having a first indicator surface an axially-extending protrusion, the main body defining a first inner bore extending therethrough, and
a second member fixed axially relative to the first member and comprising a second main body having a second indicator surface, a recess within which the axially-extending protrusion of the first member is disposed, and an annular element extending from a first end portion of the main body axially away from a second end portion of the main body and into the first inner bore, the annular element defining a second inner bore extending therethrough, the shaft extending through the second inner bore;
wherein the clamping device is movable between a first, release state in which the second inner bore has a first diameter, and a second, clamped state wherein the second inner bore has a second diameter smaller than the first diameter; and
wherein the recess comprises a first stopping surface and a second stopping surface and wherein when the first and second members are in the release state the axially-extending protrusion abuts the first stopping surface and when the first and second members are in the clamped state the axially-extending protrusion abuts the second stopping surface.

### Example 70

The assembly of any example herein, particularly example 69, wherein the first inner bore has a non-circular cross-sectional profile and wherein the annular element is received within the first inner bore and comprises an outer surface having a non-circular cross-sectional profile corresponding to the non-circular cross-sectional profile of the first inner bore.

### Example 71

The assembly of any example herein, particularly any one of examples 69-70, further comprising a tubular elastomeric member disposed within the annular element such that the shaft extends through a lumen of the elastomeric member.

### Example 72

The assembly of any example herein, particularly example 71, wherein when the clamping device is in the clamped state an inner surface of the elastomeric member frictionally engages an outer surface of the shaft.

### Example 73

A clamping device, comprising:
a first member comprising a first main body having a first indicator surface and defining a first inner bore extending through the main body, and
a second member fixed axially relative to the first member and comprising a second main body having a second indicator surface and an annular element extending from a first end portion of the main body axially away from a second end portion of the main body and into the first inner bore, the annular element defining a second inner bore extending therethrough;
wherein the clamping device is movable between a first, release state in which the second inner bore has a first diameter, and a second, clamped state wherein the second inner bore has a second diameter smaller than the first diameter; and
wherein when the clamping device is in the clamped state the first and second indicator surfaces are rotationally aligned with one another, and wherein when the clamping device is in the release state the first and second indicator surfaces are rotationally offset from one another.

### Example 74

A method, comprising:
disposing a clamping device in a release state on a shaft such that the shaft extends through the clamping device, wherein when in the release state a first indicator surface and a second indicator surface of the clamping device are rotationally offset from one another;
moving the clamping device along the shaft to a selected position; and
rotating a first member of the clamping device relative to a second member of the clamping device to move the clamping device from the release state to a clamped state such that the clamping device is restrained from movement relative to the shaft and such that the first and second indicator surfaces are rotationally aligned with one another, the first member comprising a first main body including the first indicator surface and defining a first inner bore having a non-circular cross-sectional profile, and the second member comprising a second main body including the second indicator surface and an annular element extending from a first end portion of the main body and defining a second inner bore, the annular element received within the first inner bore and comprising an outer surface having a non-circular cross-sectional profile.

### Example 75

The method of any example herein, particularly example 74, wherein rotating the first member relative to the second member moves the annular element from a rotationally aligned position relative to the first inner bore to a rotationally offset position.

### Example 76

The method of any example herein, particularly any one of examples 74-75, wherein the clamping device further comprises an elastomeric member disposed within the annular element, and wherein rotating the clamping device from the release state to the clamped state causes an inner lumen of the elastomeric member to move from a first diameter to a second, smaller diameter wherein an inner surface of the elastomeric member contacts an outer surface of the shaft.

### Example 77

A method, comprising: sterilizing the devices or assemblies of any one of claims 1-73.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

What is claimed is:

1. A clamping device for a shaft of a catheter, comprising:
a first member comprising a first main body having a first indicator surface, the first main body defining a first inner bore having a non-circular cross-sectional profile;
a second member comprising a second main body having a second indicator surface and an annular element extending from a first end portion of the main body and defining a second inner bore, the annular element received within the first inner bore and comprising an outer surface having a non-circular cross-sectional profile;
wherein the clamping device is movable between a first, release state in which the second member is in a first rotational position relative to the first member and the second inner bore has a first diameter, and a second, clamped state in which the second member is in a second rotational position relative to the first member and the second inner bore has a second diameter less than the first diameter; and
wherein when the clamping device is in the clamped state the first and second indicator surfaces are rotationally aligned with one another, and wherein when the clamping device is in the release state the first and second indicator surfaces are rotationally offset from one another.

2. The clamping device of claim 1, further comprising an elastomeric member disposed within the annular element, the elastomeric member defining an inner lumen sized to receive a catheter shaft.

3. The clamping device of claim 2, wherein when the clamping device is in the clamped state the clamping device is configured to engage an outer surface of a catheter shaft extending through the inner lumen and the clamping device is restrained from axial movement relative to the catheter shaft, and wherein when the clamping device is in the release state the clamping device can be moved axially along a length of the catheter shaft.

4. The clamping device of claim 1, wherein the first and second indicator surfaces each comprise a respective channel extending at least partially along a length of the indicator surface.

5. The clamping device of claim 1, wherein the first member comprises a third indicator surface opposite the first indicator surface and the second member comprises a fourth indicator surface opposite the second indicator surface.

6. The clamping device of claim 1, wherein the first and second members comprise a plurality of outer surfaces and wherein the outer surfaces are concave.

7. The clamping device of claim 1, further comprising a cap member coupled to the first member.

8. The clamping device of claim 7, wherein the cap member is disposed within a first end portion of the first member.

9. The clamping device of claim 7, wherein the cap member is disposed over a first end portion of the first member.

10. The clamping device of claim 1, wherein when in the clamped state, the first inner bore and the annular element are rotationally offset from one another such that the annular element is compressed by the first inner bore, thereby reducing the diameter of the second inner bore.

11. The clamping device of claim 1, wherein the annular element comprises one or more engagement members extending longitudinally along the annular element and defining the non-circular outer profile of the annular element.

12. The clamping device of claim 11, wherein when the first inner bore and the annular element are rotationally offset from one another, the engagement members are deflected inwardly toward the second inner bore.

13. The clamping device of claim 1, wherein the annular element comprises one or more openings extending longitudinally along the annular element and spaced apart about a circumference of the annular element.

14. The clamping device of claim 13, wherein when the clamping device is in the release state the openings have a first width and when the clamping device is in the clamped state the openings have a second width narrower than the first width.

15. The clamping device of claim 1, wherein the first member comprises one or more axially-extending protrusions disposed in one or more corresponding recesses of the second member, wherein the recesses each comprise a first stopping surface and a second stopping surface and wherein when the first and second members are rotated relative to one another the one or more protrusions can abut the one or more stopping surfaces to prevent rotation of the first and second members relative to one another past a selected point.

16. A catheter assembly, comprising:
a shaft; and
a clamping device disposed on the shaft, the clamping device comprising:
a first member comprising a first main body having a first indicator surface, the first main body defining a first inner bore having a non-circular cross-sectional profile, and
a second member comprising a second main body having a second indicator surface and an annular element extending from a first end portion of the main body, the annular element having a non-circular cross-sectional outer profile corresponding to the non-circular cross-sectional profile of the first inner bore, the second member defining a second inner bore extending through the annular element, the shaft extending through the second inner bore;
wherein the annular element is received within the first inner bore;
wherein the clamping device is movable between a first, release state in which the second member is in a first rotational position relative to the first member and the second inner bore has a first diameter, and a second, clamped state in which the second member is in a second rotational position relative to the first member and the second inner bore has a second diameter less than the first diameter; and
wherein when the clamping device is in the clamped state the first and second indicator surfaces are rotationally aligned with one another, and wherein when the clamping device is in the release state the first and second indicator surfaces are rotationally offset from one another.

17. The assembly of claim 16, wherein when the second member is in the first rotational position the non-circular cross-section of the annular element is rotationally aligned with the non-circular cross-section of the first inner bore, and wherein when the second member is in the second rotational position the non-circular cross-section of the annular element is rotationally offset from the non-circular cross-section of the first inner bore.

18. The assembly of claim 16, further comprising a tubular elastomeric member disposed within the annular element such that the shaft extends through a lumen of the elastomeric member, wherein when the clamping device is in the clamped state an inner surface of the elastomeric member frictionally engages the outer surface of the shaft.

19. A clamping device, comprising:
a first member comprising a first main body having a first indicator surface and an axially-extending protrusion, the main body defining a first inner bore extending therethrough;
a second member comprising a second main body having a second indicator surface, a recess within which the axially-extending protrusion of the first member is disposed, and an annular element extending from a first end portion of the main body axially away from a second end portion of the main body and into the first inner bore, the annular element defining a second inner bore extending therethrough;
wherein the clamping device is movable between a first, release state in which the second inner bore has a first diameter, and a second, clamped state wherein the second inner bore has a second diameter smaller than the first diameter; and
wherein the recess comprises a first stopping surface and a second stopping surface and wherein when the first and second members are in the release state the axially-extending protrusion abuts the first stopping surface and when the first and second members are in the clamped state the axially-extending protrusion abuts the second stopping surface.

20. The clamping device of claim 19, wherein the inner bore of the first member has a non-circular cross-sectional profile and wherein the annular element comprises an outer surface having a corresponding non-circular cross-sectional profile.

21. The clamping device of claim 19, wherein when the second member is in a first rotational position relative to the first member the clamping device is in the release state, and wherein when the second member is in a second rotational position relative to the first member the clamping device is in the clamped state.

22. The clamping device of claim 21, wherein when the second member is in the first rotational position the non-circular cross-section of the annular element is rotationally aligned with the non-circular cross-section of the inner bore of the first member, and wherein when the second member is in the second rotational position the non-circular cross-section of the annular element is rotationally offset from the non-circular cross-section of the inner bore of the first member.

23. The clamping device of claim 19, further comprising an elastomeric member disposed within the annular element, the elastomeric member defining an inner lumen sized to receive a catheter shaft.

24. The clamping device of claim 19, wherein when the clamping device is in the clamped state the clamping device is configured to engage an outer surface of a catheter shaft extending through the second inner bore and the clamping device is restrained from axial movement relative to the catheter shaft, and wherein when the clamping device is in the release state the clamping device can be moved axially along a length of the catheter shaft.
